(19) United States
(12) Patent Application Publication
(10) Pub. No.: US 2025/0054577 A1
(43) Pub. Date: Feb. 13, 2025

(54) SYSTEM FOR EVALUATING THE QUALITY OF MESENCHYMAL STROMAL CELLS

(71) Applicant: TASLY STEM CELL BIOLOGY LABORATORY, TASLY GROUP, LTD., Tianjin (CN)

(72) Inventors: Yujian James KANG, Tianjin (CN); Jinlai ZHANG, Tianjin (CN); Fei MA, Tianjin (CN)

(73) Assignee: TASLY STEM CELL BIOLOGY LABORATORY, TASLY GROUP, LTD., Tianjin (CN)

(21) Appl. No.: 18/718,007

(22) PCT Filed: Dec. 16, 2022
(86) PCT No.: PCT/CN2022/139595

§ 371 Date: Jun. 7, 2024

(30) Foreign Application Priority Data
Jan. 14, 2022 (CN) 202210043060.6

(51) Int. Cl. G16B 25/10; C12Q 1/6874; C12Q 1/6876; G06N 20/00

(52) U.S. Cl. CPC G16B 25/10; C12Q 1/6874; C12Q 1/6876; G06N 20/00; C12Q 2600/158

(57) ABSTRACT

The present disclosure provides a system for evaluating the quality of stem cells, comprising: a quality scoring module, used for calculating the quality score of the stem cells based on the expression level of feature genes related with the quality of stem cells and weight coefficient of the feature genes; a quality evaluation module, used for evaluating the quality of the stem cells based on the quality score of the stem cells; and a result output module, used for outputting a result report of the quality of the stem cells; further comprising: a subpopulation clustering module, used for obtaining single-cell gene expression data and specific quality attributes of the stem cells; a subpopulation identification module, used for determining a quality predictive model of the stem cells, the feature genes related with the quality of stem cells, and weight coefficient of the feature genes based on the single-cell gene expression data and the specific quality attributes of the stem cells. The present disclosure achieves the effect of accurately and quantitatively evaluating the quality of stem cells, and the system can be used for screening the stem cells with high quality.

### CROSS REFERENCE TO RELATED APPLICATION

This application claims priority of Chinese application No. 202210043060.6, filed on Jan. 14, 2022, the entire content of which is incorporated herein by reference.

### FIELD

The present disclosure relates to the technical field of mesenchymal stromal cells, and relates to a system for evaluating the quality of mesenchymal stromal cells.

### BACKGROUND

Cell therapy is the future of medicine, which is expected to fundamentally change the clinical dilemma of currently-untreatable diseases faced by existing medicine by restoring tissue function and treating the root cause of degenerative diseases.

A necessary prerequisite to support cell research and application is to obtain sufficient cells by moderate expansion. However, diverse microenvironment causes gene expression changes and heterogeneity of mesenchymal stromal cell (MSC) from the same origin during the process of propagation. Such heterogeneity seriously hinders MSC scientific research and constitutes fatal risks for MSC clinical application. Therefore, identifying heterogeneity in MSC amplification is a key prerequisite for the clinical development of MSC therapy.

Single-cell RNA sequencing (scRNA-seq) provides possibility to explore the heterogeneity among cells, which can preliminarily analyze the heterogeneity of cell subpopulations based on gene expression profiles, but cannot clarify the relationship between the heterogeneity and the quality of the cells, and cannot determine the quality of the MSCs quantitatively.

CN113061638A provides a system for evaluating stem cells, which performs sterility detection, safety detection, cell activity detection and cell morphology detection on stem cells.

However, the existing technology lacks a sound and unified norm and standard for evaluating the quality of the MSCs, cannot accurately reveal the influence of the microenvironment on MSCs. The safety issue of MSCs therapy remains unaddressed. The industry of MSCs faces unprecedented challenge of imperfect quality control system, incomplete mechanism research, and non-standard clinical application.

### SUMMARY

In view of the deficiencies and actual needs of the prior art, the present disclosure provides a system for evaluating the quality of mesenchymal stromal cells, which determines a key quality classification standard of mesenchymal stromal cells at a single-cell level. Based on the single-cell transcriptomic analysis and functional clustering method of cell subpopulation, a single-cell gene expression dataset of mesenchymal stromal cells with quality attribute labels is obtained. A quality predictive model of mesenchymal stromal cells is constructed using a supervised machine learning method, which determines feature genes related with the quality of mesenchymal stromal cells and weight coefficient of the feature genes. The quality risk caused by the heterogeneity of mesenchymal stromal cells is quantitatively determined.

A first aspect of the present disclosure provides a system for evaluating the quality of mesenchymal stromal cells, comprising:
an evaluation subsystem, which comprises:
a quality scoring module, used for calculating the quality score of the mesenchymal stromal cells, based on the expression level of feature genes related with the quality of the mesenchymal stromal cells and weight coefficient of the feature genes; and
a quality evaluation module, used for evaluating the quality of the mesenchymal stromal cells based on the quality score of the mesenchymal stromal cells.

The mesenchymal stromal cells in clinic have the same cell biological properties, but will develop heterogeneity under the influence of the microenvironment. In order to uncover cellular heterogeneity, predict cell state/fate and evaluate the quality of the mesenchymal stromal cells, the feature genes related with the quality of mesenchymal stromal cells and weight coefficient of the feature genes are determined by using bioinformatics means, and the quality of the mesenchymal stromal cells is evaluated based on the expression level of the feature genes and weight coefficient of the feature genes.

In the present disclosure, using a supervised machine learning model to learn the single-cell gene expression dataset with quality attribute labels, the feature genes related with the quality of mesenchymal stromal cells which can accurately define the differences of different mesenchymal stromal cells, and the weight coefficient of the feature genes are determined. The quality score of mesenchymal stromal cells is calculated to evaluate the quality of the mesenchymal stromal cells quantitatively, based on the expression level of feature genes of the mesenchymal stromal cell samples to be tested and the weight coefficient of the feature genes.

Preferably, the quality scoring module comprises: (1) a unit for obtaining the expression level of the feature genes, used for obtaining the expression level of the feature genes related with the quality of the mesenchymal stromal cells; (2) a calculation unit, used for calculating the quality score of the mesenchymal stromal cells based on the expression level of the feature genes and the weight coefficient of the feature genes;
the function for calculating the quality score of the mesenchymal stromal cells is:

$$quality score = 1 + {e}^{- {\sum }_{i = 1}^{n} {W}_{i} * {G}_{i}}$$

Gi is the expression level of the ith feature gene in single mesenchymal stromal cell, Wi is the weight coefficient of the ith feature gene, and n is the number of the feature genes.

Preferably, the method for obtaining expression level of feature genes includes conventional gene quantification methods in the art, such as single-cell sequencing, high-throughput sequencing, microarray chip, qPCR, etc., and preferably the single-cell sequencing is used to obtain the expression level of feature genes.

Preferably, the quality evaluation module comprises: (1) a unit for determining a quality risk threshold of the mesenchymal stromal cells, used for analyzing the quality score of the mesenchymal stromal cells of a dataset using receptor operating characteristic curve and area under the curve, a value of the highest point of the receptor operating characteristic curve is the quality risk threshold of the mesenchymal stromal cells; wherein, the dataset contains the single-cell gene expression data of the mesenchymal stromal cells with known specific quality attribute labels; (2) a comparison and judgment unit, used for comparing the quality score of the mesenchymal stromal cells and the quality risk threshold,
if the quality score of the mesenchymal stromal cells≥the quality risk threshold of the mesenchymal stromal cells, the mesenchymal stromal cells are the mesenchymal stromal cells with quality risk;
if the quality score of the mesenchymal stromal cells<the quality risk threshold of the mesenchymal stromal cells, the mesenchymal stromal cells are the mesenchymal stromal cells without quality risk.

Preferably, the evaluation subsystem further includes:
a result output module, used for outputting a result report by the quality evaluation module of the mesenchymal stromal cells.

A second aspect of the present disclosure provides an optimization subsystem, which comprises:
a subpopulation clustering module, used for obtaining single-cell gene expression data and specific quality attributes of the mesenchymal stromal cells;
a subpopulation identification module, used for determining and/or optimizing a quality predictive model of the mesenchymal stromal cells, the feature genes related with the quality of the mesenchymal stromal cells, and weight coefficient of the feature genes based on the single-cell gene expression data and the specific quality attributes of the mesenchymal stromal cells.

Preferably, the subpopulation clustering module comprises: (1) a unit for obtaining the single-cell gene expression data, used for preprocessing single-cell RNA sequencing data, to obtain the single-cell gene expression data of the mesenchymal stromal cells; (2) a unit for obtaining a pathway score matrix, used for performing pathway enrichment analysis on the single-cell gene expression data of the mesenchymal stromal cells, and calculating the pathway score in each mesenchymal stromal cell for each pathway to which the gene is enriched to obtain the score matrix of single-cell pathway enrichment of the mesenchymal stromal cells; (3) a unit for determining the specific quality attribute, used for performing normalization, dimensional reduction, clustering and visual process on the pathway score matrix of the single-cell pathway enrichment of the mesenchymal stromal cells to obtain a clustering result of single-cell subpopulations of the mesenchymal stromal cells as specific quality attributes of the mesenchymal stromal cells.

In the present disclosure, a pathway score matrix is established by integrating traditional cell subpopulation clustering, differential gene analysis and pathway enrichment analysis. Each column of the pathway score matrix represents the expression of a pathway in different mesenchymal stromal cells, each row represents cells indices, and the data in each grid represents the expression of a specific pathway in a specific mesenchymal stromal cell. The functional clustering method based on the pathways achieves the effect of rapidly discovering functional differences in mesenchymal stromal cells.

Preferably, the subpopulation identification module comprises: (1) a unit for establishing a dataset, used for forming a dataset with the single-cell gene expression data of the mesenchymal stromal cells with specific quality attribute labels; (2) a unit for dividing the dataset, used for classifying the dataset as training set and test sets; (3) a model training unit, used for determining and/or optimizing the quality predictive model of the mesenchymal stromal cells using the training set to train a supervised machine learning model, and adjusting parameters of the supervised machine learning model by cross-validation and test sets; (4) a unit for outputting the feature genes, used for outputting the feature genes related with the quality of the mesenchymal stromal cells and weight coefficients based on the quality predictive model of the mesenchymal stromal cells.

In the present disclosure, the subpopulation identification module of the system for evaluating the quality of mesenchymal stromal cells performs the function, wherein the supervised machine learning model is trained by using the single-cell gene expression data of the mesenchymal stromal cells with known quality attribute labels as the dataset, which are randomly classified as training set and test sets in a certain ratio: using the training set to determine the number of characteristics of the supervised machine learning model, using the test sets to adjust parameters and optimize the supervised machine learning model, and obtaining a model that has good performance in test accuracy, precision, recall and F1 score as a model for predicting the quality of the mesenchymal stromal cells.

Preferably, the supervised machine learning model comprises any of a perceptron model, a K-nearest neighbor algorithm, a naive Bayesian model, a decision tree model, logical regression, a support vector machine, random forest, a boosting method model, an EM algorithm or conditional random field.

Preferably, the mesenchymal stromal cells include any one or a combination of at least two of adult mesenchymal stromal cells, embryonic mesenchymal stromal cells, induced pluripotent mesenchymal stromal cells or mesenchymal stromal cells transformed by mature somatic cells and the derived cells thereof.

Preferably, the mesenchymal stromal cells include any one or a combination of at least two of mesenchymal stromal cells, mesenchymal stromal cells, multipotent stromal cells, multipotent mesenchymal stromal cells or medicinal signaling cells.

Preferably, the mesenchymal stromal cells include any one or a combination of at least two of adipose-derived mesenchymal stromal cells, umbilical cord mesenchymal stromal cells, placenta-derived mesenchymal stromal cells, bone marrow mesenchymal stromal cells, dental pulp mesenchymal stromal cells, menstrual blood-derived mesenchymal stromal cells, amniotic epithelial mesenchymal stromal cells, bronchial basal layer cells.

The present disclosure also provides a method for optimizing the system for evaluating the quality of mesenchymal stromal cells, including:
preprocessing the newly obtained single-cell RNA sequencing data by the subpopulation clustering module of the optimization subsystem to obtain single-cell gene expression data; performing pathway enrichment analysis on the single-cell gene expression data and calculating pathway score in each mesenchymal stromal cell to obtain the pathway score matrix; and performing normalization, dimensional reduction, clustering and visual process on the pathway score matrix to obtain a clustering result as specific quality attributes of the mesenchymal stromal cells.

The subpopulation identification module of the optimization subsystem integrates the single-cell gene expression data of mesenchymal stromal cells with specific quality attribute labels into the original dataset to form a new dataset, which is classified as training set and test sets; optimizing the quality predictive model of the mesenchymal stromal cells using the training set to train supervised machine learning model, and adjusting parameters of the supervised machine learning model by cross-validation and testing with the test set; outputting the feature genes related with the quality of the mesenchymal stromal cells and weight coefficients.

The quality scoring module of the evaluation subsystem obtains the expression of the feature genes related with the quality of the mesenchymal stromal cells, and calculates quality score of the mesenchymal stromal cells based on the expression and the weight coefficient of the feature genes.

The function for calculating the quality score of the mesenchymal stromal cells is:

quality score=1+e^{-Σ}^{ᵢ₌₁}^{ⁿ}^{w}^{ᵢ}^{*G}^{ᵢ}

Gi is the expression level of the ith feature gene, Wi is the weight coefficient of the ith feature gene, and n is the number of the feature genes.

The quality evaluation module of the evaluation subsystem compares the quality score of the mesenchymal stromal cells and the quality risk threshold, wherein, the dataset contains the single-cell gene expression data of the mesenchymal stromal cells with known specific quality attribute labels;
if the quality score of the mesenchymal stromal cells≥the quality risk threshold of the mesenchymal stromal cells, the mesenchymal stromal cells are the mesenchymal stromal cells with quality risk;
if the quality score of the mesenchymal stromal cells<the quality risk threshold of the mesenchymal stromal cells, the mesenchymal stromal cells are the mesenchymal stromal cells without quality risk.

A third aspect of the present disclosure provides a method for establishing a quality predictive model of mesenchymal stromal cells, comprising:
obtaining single-cell gene expression data with specific quality attributes of the mesenchymal stromal cells to form a dataset, which is classified as training set and test sets;
determining a quality predictive model of mesenchymal stromal cells by using the training set to train a supervised machine learning model and adjusting parameters of the supervised machine learning model by cross-validation and testing with the test sets.

Preferably, the method for obtaining single-cell gene expression data with specific quality attributes of the mesenchymal stromal cells comprises:
obtaining single-cell gene expression data of the mesenchymal stromal cells by single-cell RNA sequencing of the mesenchymal stromal cells;
obtaining a pathway score matrix by pathway enrichment analysis on the single-cell gene expression data of the mesenchymal stromal cells and calculating a pathway enrichment score in each of the mesenchymal stromal cells;
obtaining a clustering result of the mesenchymal stromal cells as the specific quality attributes of the mesenchymal stromal cells by bioinformatic analysis on the pathway score matrix.

Preferably, the bioinformatic analysis on the pathway score matrix of the mesenchymal stromal cells includes:
performing dimensional reduction and clustering on the pathway score matrix.

Preferably, the establishing method further includes:
determining the feature genes related with the quality of mesenchymal stromal cells and the weight coefficient of the feature genes based on the quality predictive model of the mesenchymal stromal cells.

Compared with the prior art, the present disclosure has the following beneficial effects:
(1) The system for evaluating the quality of mesenchymal stromal cells of the present disclosure uses quality scoring module and quality evaluation module to accurately and quantitatively evaluate the quality of mesenchymal stromal cell, based on the determined feature genes related with the quality of the mesenchymal stromal cells and the weight coefficient of the feature genes. According to the weighted sum of the feature genes related with the quality of the mesenchymal stromal cells and the quality risk threshold, the system outputs mesenchymal stromal cell quality, which is a standardized, comprehensive and unified system for evaluating the quality of the mesenchymal stromal cells.
(2) The system for evaluating the quality of mesenchymal stromal cells of the present disclosure uses subpopulation clustering module and subpopulation identification module to continuously update the quality standard map of the mesenchymal stromal cells at the single-cell level based on the accumulated single-cell RNA sequencing data of the mesenchymal stromal cells. Using this mesenchymal stromal cell quality standard map as a dataset, the quality predictive model of the mesenchymal stromal cells is optimized based on a supervised machine learning model to improve the accuracy of quality evaluation results of the mesenchymal stromal cells.
(3) The system for evaluating the quality of mesenchymal stromal cells of the present disclosure achieves the effect of accurately and quantitatively uncovering cellular heterogeneity of mesenchymal stromal cells under the influence of the microenvironment.
(4) The system for evaluating the quality of mesenchymal stromal cells of the present disclosure can be used to screen the mesenchymal stromal cells with high quality.

### BRIEF DESCRIPTION OF THE DRAWINGS

FIG. 1A is the growth curve of D1M1-P5; FIG. 1B is the growth curve of D1M2-P5; FIG. 1C is the cell cycle analysis of D1M1-P5; FIG. 1D is the cell cycle analysis of D1M2-P5; FIG. 1E is the apoptosis population of D1M2-P5; FIG. 1F is the apoptosis population of D1M2-P5; FIG. 1G is the adipogenic differentiation, osteogenic differentiation and chondrogenic differentiation of D1M1-P5; FIG. 1H is the adipogenic differentiation, osteogenic differentiation and chondrogenic differentiation of D1M2-P5.

FIG. 2A shows the lung tissues and HE staining results of mice post infusion of D1M1-P5, D1M2-P5 or saline; Black arrows indicate phlebothrombosis; FIG. 2B shows the density of emboli found in each 10× visual field; *p<0.05; FIG. 2C shows the fluorescent results in the lungs of mice infused with D1M1-P5, D1M2-P5 or saline; FIG. 2D shows the number of PKH26+ cells found in each 10× visual field.

FIG. 3A shows the clustering results of cell subpopulation of D1M1-P5 and D1M2-P5, where 0, 1, 2, 3, 4, and 5 represent different mesenchymal stromal cell clusters; FIG. 3B is the expression of risk genes by different mesenchymal stromal cell subpopulation based on the GO-BP database, where C0, C1, C2, C3, C4, and C5 (corresponding to the mesenchymal stromal cell cluster 0, 1, 2, 3, 4, and 5 in FIG. 3A) represent different mesenchymal stromal cell clusters, respectively; FIG. 3C shows the expression of risk genes by different mesenchymal stromal cell subpopulation based on the KEGG database, where C0, C1, C2, C3, C4, and C5 (corresponding to the mesenchymal stromal cell clusters 0, 1, 2, 3, 4, and 5 in FIG. 3A) represent different mesenchymal stromal cell clusters.

FIG. 4 is a schematic diagram of functional clustering procedure.

FIG. 5A shows the functional clustering results of cell subpopulation obtained by using the ssGSEA scoring function, where A2105C2P5 (ie DIM1-P5) are the mesenchymal stromal cells with quality risk, and A2105C3P5 (ie D1M2-P5) are the mesenchymal stromal cells without quality risk; FIG. 5B is the functional clustering results of cell subpopulation obtained by using the AUCell scoring function, where A2105C2P5 (ie D1M1-P5) are the mesenchymal stromal cells with quality risk, and A2105C3P5 (ie D1M2-P5) are the mesenchymal stromal cells without quality risk; FIG. 5C shows the functional clustering results of cell subpopulation obtained by using the Seurat scoring function, where A2105C2P5 (ie D1M1-P5) are the mesenchymal stromal cells with quality risk, and A2105C3P5 (ie D1M2-P5) are the mesenchymal stromal cells without quality risk.

FIG. 6 is a schematic diagram of cross-culture of mesenchymal stromal cells.

FIG. 7A shows the functional clustering results of cell subpopulation obtained by using the ssGSEA scoring function, where D1M1-P3, D1M1-P5, D1M2/M1-P5 are the mesenchymal stromal cells with quality risk, and D1M2-P3, D1M2-P5, D1M1/M2-P5 are the mesenchymal stromal cells without quality risk; FIG. 7B shows the functional clustering results of cell subpopulation obtained by using the AUCell scoring function, where D1M1-P3, D1M1-P5, D1M2/M1-P5 are the mesenchymal stromal cells with quality risk, and D1M2-P3, D1M2-P5, D1M1/M2-P5 are the mesenchymal stromal cells without quality risk; FIG. 7C shows the functional clustering results of cell subpopulation obtained by using the Seurat scoring function, where D1M1-P3, D1M1-P5, D1M2/M1-P5 are the mesenchymal stromal cells with quality risk, and D1M2-P3, D1M2-P5, D1M1/M2-P5 are the mesenchymal stromal cells without quality risk.

FIG. 8A shows the lung tissues and HE staining results of mice post infusion of D1M1-P3, D1M2-P3, D1M2/M1-P5, D1M2/M1-P5 or saline; Black arrows indicate phlebothrombosis; FIG. 8B shows the density of emboli found in each 10× visual field; *p<0.05.

FIG. 9 is a schematic diagram of constructing a quality predictive model of mesenchymal stromal cells.

FIG. 10 shows variation curve of the cross-validation accuracy with an increase of the gene number during the recursive feature elimination (RFE) process, and zoom in of the turning point on eight different RFE variation curves (M1, M2, M3, M4, M5, M6, M7, and M8).

FIG. 11 is the quality score thresholds with the highest sensitivity and specificity in classifying mesenchymal stromal cells with quality risk or without quality risk in four test datasets.

FIG. 12A is the density distribution of quality score of mesenchymal stromal cells in test set 1; FIG. 12B is the density distribution of quality score of mesenchymal stromal cells in test set 2; FIG. 12C is the density distribution of quality score of mesenchymal stromal cells in test set 3; FIG. 12D is the density distribution of quality score of mesenchymal stromal cells in test set 4.

FIG. 13A is a schematic diagram of the evaluation subsystem 10 of the system for evaluating the quality of mesenchymal stromal cells, in which, 110—quality scoring module, 120—quality evaluation module; FIG. 13B is a schematic diagram of the quality scoring module 110, in which, 1110—unit for obtaining expression of feature genes, 1120—calculation unit; FIG. 13C is a schematic diagram of the quality evaluation module 120, in which, 1210—unit for determining quality risk threshold, 1220—comparison and judgment unit; FIG. 13D is a schematic diagram of the optimization subsystem 20 of the system for evaluating the quality of mesenchymal stromal cells, in which, 210—subpopulation clustering module, 220—subpopulation identification module; FIG. 13E is a schematic diagram of the subpopulation clustering module 210, in which, 2110—unit for obtaining single-cell gene expression data, 2120—unit for obtaining pathway score matrix, 2130—unit for determining specific quality attribute; FIG. 13F is a schematic diagram of the subpopulation identification module 220, in which, 2210—unit for establishing dataset, 2220—unit for dividing dataset, 2230—model training unit, 2240—unit for outputting feature genes; FIG. 13G is a schematic diagram of the system for evaluating the quality of mesenchymal stromal cells, in which, 10—evaluation subsystem, 20—optimization subsystem.

FIG. 14 is the prediction of the quality of D1M1/M2-P5 and D1M2/M1-P5 by the feature genes determined by the quality predictive model of mesenchymal stromal cells and the weight coefficient of the feature gene.

### DETAILED DESCRIPTION

In order to further illustrate the technical means adopted by the present disclosure and its effects, the present disclosure will be further described below with reference to the embodiments and accompanying drawings. It should be understood that the specific embodiments described herein are only used to explain the present disclosure, but not to limit the present disclosure. Various modifications or variations of the methods and systems of the present disclosure will be apparent to those skilled in the art without departing from the scope and spirit of the present disclosure. Although the present disclosure has been described in connection with certain preferred embodiments, it is to be understood that the present disclosure, as claimed, should not be unduly limited to these particular embodiments, and various modifications and additions should be made to the described embodiments within the scope of the present disclosure. Certainly, various modifications made to the described embodiments by those skilled in molecular biology and related fields in order to implement the present disclosure fall within the protection scope of the claims.

If no specific technique or condition is written in the embodiments, the technique or condition described in the literature in the field or the product specification is used. The reagents or instruments used without the manufacturer's indication are conventional products that can be purchased through regular channels.

### Definition

As used in the context, “mesenchymal stromal cell” refers to one kind of cells which is relatively undifferentiated, has the potential to differentiate, and can actively divide and circulate, producing appropriate stimuli for mature, differentiated, and functional cell lines. The properties defined for the mesenchymal stromal cells include: (a) the mesenchymal stromal cells are not terminally differentiated by themselves; (b) they can divide indefinitely throughout the life of the animal; (c) they have the consistent characterization results by cell markers, and are a type of mesenchymal stromal cells, not several types of mesenchymal stromal cells and/or a mixture of somatic cells; (d) when the mesenchymal stromal cells divide, each daughter cell can remain as a mesenchymal stromal cell or carry out a process that irreversibly leads to terminal differentiation.

As described in the context, “multipotent mesenchymal stromal cells” are pluripotent mesenchymal stromal cells that can differentiate into several types of cells. Multipotent mesenchymal stromal cells have been shown to differentiate into cell types in vitro or in vivo, including osteoblasts, chondrocytes, myocytes and adipocytes. Mesenchyme is embryonic connective tissue which is derived from mesoderm and differentiated into hematopoietic tissue and connective tissue, in which multipotent mesenchymal stromal cells do not differentiate into hematopoietic cells.

As described in the context, “the quality of the mesenchymal stromal cells” refers to any of the above-mentioned factors related with the safety of mesenchymal stromal cells. Mesenchymal stromal cells develop heterogeneity under the influence of the microenvironment, and the quality risk possibility results from such heterogeneity. Clinical-grade mesenchymal stromal cells contain one type of mesenchymal stromal cells, not several types of mesenchymal stromal cells and/or a mixture of mesenchymal stromal cells and somatic cells, which should be tested strictly by a third party and laboratory, including cell viability, biological function, tumorigenicity, embolism, immunogenicity, microorganisms, mycoplasma, endotoxin testing, etc, which are closely related with the safety, efficacy and consistency of mesenchymal stromal cells. A release test is required by qualified mesenchymal stromal cells before their transplantation, to further perform a conformity test with microorganisms, mycoplasma and endotoxin, avoiding acute or subacute serious adverse reactions during or after transplantation, such as fever, allergy, bacteriaemia, etc.

As used in the context, “feature genes related with the quality of the mesenchymal stromal cells” refer to genes that determine the quality category of the mesenchymal stromal cells. When the expression of the genes increases, the quality risk of mesenchymal stromal cells will increase or decrease.

As used in the context, “expression level” refers to the expression level of a gene.

As mentioned in the context, “quality score of mesenchymal stromal cells” refers to the score calculated according to the following function, based on the expression level of feature genes and weight coefficient of each feature gene determined by the quality predictive model of the mesenchymal stromal cells;

$$quality score = 1 + {e}^{- {\sum }_{i = 1}^{n} {W}_{i} * {G}_{i}}$$

Gi is the expression level of the ith feature gene in single mesenchymal stromal cell, Wi is the weight coefficient of the ith feature gene, and n is the number of the feature genes.

As described in the context, “gene expression level” refers to the expression level of a specific gene in a cell, which is measured using the conventional methods in the field of molecular biology. For example, it includes the hybridization level value (measurement data) in the form of fluorescence intensity which is determined between probe nucleic acids immobilized on the surface of the DNA chip plate, the estimated value of gene expression level obtained based on the numerical value, and the like.

As used in the context, “specific quality attributes” refer to the clustering results of subpopulation of single mesenchymal stromal cell determined using subpopulation clustering methods, i.e. “mesenchymal stromal cells with quality risk” or “mesenchymal stromal cells without quality risk”.

As mentioned in the context, the pathway score matrix is with pathway identities as columns and cell indices as rows, and the data in each grid represents the expression of a specific pathway in a specific mesenchymal stromal cell. The method for analyzing data (including supervised and unsupervised data analysis and bioinformatics methods) is disclosed in Brazma and ViIo J, 2000, FEBS Lett 480(1):17-24.

Examples of the mesenchymal stromal cell-induced embolism risk closely related with the quality of the mesenchymal stromal cells are described in the following embodiments.

Those skilled in the art will understand that, according to the present disclosure, the tumorigenicity and immunogenicity of mesenchymal stromal cells can be identified using substantially the same methods and means. For example, the feature genes related with tumorigenicity can be c-myc; the feature genes related with immunogenicity can be dnam-1, mcp-1.

The mesenchymal stromal cell-induced embolism risk is the most typical risk of mesenchymal stromal cell application and one of the most important factors affecting the quality of mesenchymal stromal cells. In the past 20 years, many clinical cases have been reported to have embolic complications after mesenchymal stromal cell therapy (Woodard, J. P. et al. Pulmonary cytolytic thrombi: a newly recognized complication of stem cell transplantation. Bone Marrow Transpl 25, 293-300 (2000); Tatsumi, K. et al. Tissue factor triggers procoagulation in transplanted multipotent mesenchymal stem cells leading to thromboembolism. Biochem Biophys Res Commun 431, 203-209 (2013)), which indicates that those skilled in the art understand that the evaluation of this risk can be used to evaluate the quality of mesenchymal stromal cells.

### Embodiment 1. Acquisition and Culturing of Multipotent Mesenchymal Stromal Cells

### 1. Acquisition of Multipotent Mesenchymal Stromal Cells

### \textcircled{1} Collection of Adipose Tissue

In a sterile environment, the adipose tissue from donor (negative for HIV, hepatitis B virus, hepatitis C virus, human T-cell virus, Epstein-Barr virus, cytomegalovirus, and Treponema pallidum) is collected.

50-150 mL of adipose tissue is put into a closed container pre-filled with 100 mL of tissue preservation solution (purchased from TIAN JIN HAO YANG BIOLOGICAL MANUFACTURE Co., Ltd.), and stored at 2-8° C. for later use.

30 mL of tissue preservation solution is drawn with a pipette, and tested to determine if it is contaminated by bacterial, endotoxin and mycoplasma. Then the tissue is used for multipotent mesenchymal stromal cells isolation.

### \textcircled{2} Isolation of Multipotent Mesenchymal Stromal Cells

An equal volume of Dulbecco's phosphate buffered solution (dPBS) is added to the adipose tissue. The container containing the tissue is sealed, and shaken vigorously for 20 s and stands for 5 min. After the adipose tissue and dPBS are completely layered, the bottom liquid layer is discarded, and the adipose tissue is rinsed repeatedly with dPBS, until the bottom liquid is not red again.

20 mL of aliquots of the washed adipose tissue are added to 50 mL centrifuge tubes, and an equal volume of dPBS is added, centrifuged at 400 g for 5 min. The solution is divided into upper lipid layer, middle adipose tissue layer, lower dPBS and blood cell precipitate. The upper lipid layer, lower dPBS and blood cell precipitate are removed.

Twice the volume of 1 mg/mL type I collagenase (purchased from Gibco, Cat #17100-017) is added to the adipose tissue. The container containing the tissue is sealed, and transferred to a preheated thermostatic air shaker at 37° C. The tissue is digested with collagenase at 120 rpm/min for 1 h.

### \textcircled{3} Collection of Multipotent Mesenchymal Stromal Cells

The digested tissue is centrifuged at 500 g for 8 min at room temperature. After centrifugation, it is divided into upper lipid layer, middle adipose tissue layer, lower digestion solution layer and bottom cell precipitate. The upper lipid layer, middle adipose tissue layer and lower digestion solution layer are discarded. The bottom cell precipitate is resuspended with dPBS, filtered through a 100 μm filter and centrifuged at 500 g for 5 min in a 50 mL centrifuge tube. The supernatant is removed to obtain cell precipitate containing primary human adipose-derived stromal cells (hADSCs).

A complete medium equal to the volume of the adipose tissue is added to the centrifuge tube, and mixed evenly to fully dissociate the digested cells. Cell suspension containing primary hADSCs is obtained.

2. Culturing of Multipotent Mesenchymal Stromal Cells The primary hADSCs are cultured in different media M1 (αMEM+10% FBS, αMEM purchased from Thermo Fisher, FBS purchased from ExCell Bio) or M2 (DMEM/F-12+5% Helios UltraGRO-Advanced, DMEM/F-12 purchased from Thermo Fisher, Helios UltraGRO-Advanced purchased from Helios BioScience), the specific steps of which are as follows:

### \textcircled{1} Primary Culturing

The cell precipitate is resuspended in M1 medium/M2 medium, and 1.5 mL of the cell suspension is seeded into a T75 cell culture flask pre-added with 8.5 mL of M1 medium/M2 medium.

The T75 cell culture flask is labeled and transferred to a cell culture incubator, and cultured at 37° C. and 5% CO₂. After 24 hours, the primary hADSCs have basically adhered to the wall. The supernatant is removed and 10 mL of M1 medium/M2 medium is added. The medium is changed every three days thereafter.

Under the microscope, in addition to the primary hADSCs, there are many heterocytic cells and matrix components in the obtained primary cells, and hADSCs have a typical long spindle shape.

### \textcircled{2} Subculturing

When the confluence of the primary hADSCs reaches 50%-70%, the medium is removed, and the cells are washed once with 10 mL dPBS. 1.5 mL of digestion solution Tryple™-Express (1×) (purchased from Gibco, Cat #12604-021) is added for 1 to 2 min. After some cells become round and fall off, the culture flask is tapped lightly and 4.5 mL dPBS is added to stop the digestion.

The liquid is collected into a 50 mL centrifuge tube. After washed once with 10 mL dPBS, it is centrifuged at 400 g for 5 min. The upper layer is a mixture of digestion solution and dPBS, and the lower white precipitate is the precipitate containing primary hADSCs. The supernatant is removed, and the white precipitates in several centrifuge tubes are collected in one centrifuge tube and resuspended with M1 medium/M2 medium by 30 mL. Then the cell suspension is mixed evenly for cell counting. The counted cells are resuspended with M1 medium/M2 medium, and passaged at 5000-6000 cells/cm² density.

The cell culture flask is labeled with information such as cell batch, passage number, and culture time, and placed in a cell culture incubator. When the cell confluence reaches about 90%, the cells are passaged again. P3 and P5-generation multipotent mesenchymal stromal cells are collected and cryopreserved, which are named as D1M1-P3 (representing the multipotent mesenchymal stromal cells obtained by subculture of primary multipotent mesenchymal stromal cells from donor 1 in M1 medium to the P3 generation), D1M2-P3 (representing the multipotent mesenchymal stromal cells obtained by subculture of primary multipotent mesenchymal stromal cells of donor 1 in M2 medium to P3 generation), D1M1-P5 (representing the multipotent mesenchymal stromal cells obtained by subculture of primary multipotent mesenchymal stromal cells of donor 1 in M1 medium to P5 generation), D1M2-P5 (representing the multipotent mesenchymal stromal cells obtained by subculture of primary multipotent mesenchymal stromal cells of donor 1 in M2 medium to P5 generation).

### Embodiment 2. Quality Control of Multipotent Mesenchymal Stromal Cells

In this embodiment, frozen samples were rapidly thawed in a water bath at 37° C. in continuous agitation, multipotent mesenchymal stromal cells were resuspended in pre-warm dPBS, centrifuged at 400 g for 5 min and washed twice with dPBS. Finally, multipotent mesenchymal stromal cells (D1M1-P3, D1M2-P3, D1M1-P5, D1M2-P5) were counted and used in quality control.

### 1. Microbiological Safety Test

### \textcircled{1} Sterility Test

According to the Chinese Pharmacopoeia 2020 edition (volume IV) General Chapter <1101>, Sterility Test, the brief steps are as follows:

100 mL of 0.9% saline (purchased from SJZ No. 4 Pharmaceutical) is used to filter and wet the filter membrane of a disposable triple germs collector (purchased from Zhejiang Tailin Bioengineering Co., Ltd.). Then each of the hADSCs samples is introduced into the germs collector and filtered. After filtration, the filter membrane is washed twice with 300 mL of 0.9% saline.

Two different media were used: Fluid Thioglycollate Medium, to detect anaerobic and aerobic bacteria, and Tryptic Soy Broth (TSB), which is a soybean casein digest medium to detect fungi and aerobic bacteria. 100 mL of Fluid Thioglycollate Medium is added separately to two of the three incubators containing the sample in triple germs collector, and 100 mL of TSB is added to the other incubator.

The sample is replaced by 1 mL of 0.9% saline as a negative control, and Staphylococcus aureus (less than 100 CFU of added bacteria amount) is used as a positive control.

After inoculation, these media are shaken gently, and incubated under the conditions recommended for sterility tests: one Fluid Thioglycollate Medium at 30-35° C. and the others at 20-25° C., cultured for 14 days. After co-cultured for 14 days, the growth of bacteria is observed and recorded every working day during the culture period.

\textcircled{2} Mycoplasma Detection According to the Chinese Pharmacopoeia 2020 edition (volume IV) General Chapter <3301>, Mycoplasma Inspection Method, the brief steps are as follows:

Mycoplasma broth medium, mycoplasma broth medium containing arginine, mycoplasma semi-fluid medium and mycoplasma semi-fluid medium containing arginine are prepared and sterilized according to conventional recipes. Then, 800,000 units of Penicillin Sodium For Injection (purchased from Jiangxi Dongfeng Pharmaceutical Co., Ltd.) are reconstituted with 1 mL of 0.9% saline for future use. 200 mL of fetal bovine serum and 800,000 units of Penicillin Sodium For Injection are added to each of 800 mL sterilized medium, mixed well and stored at 2-8° C.

4 bottles of mycoplasma broth medium (10 mL/bottle), 4 bottles of mycoplasma broth medium containing arginine (10 mL/bottle), 2 bottles of mycoplasma semi-fluid medium (10 mL/bottle), and 2 bottles of mycoplasma semi-fluid medium containing arginine (10 mL/bottle) are inoculated with 1.0 mL of cell samples, and cultured at 36±1° C. for 21 days, and observed every 3 days.

On the 7th day after inoculation, 2 bottles of mycoplasma broth medium inoculated with cell samples and 2 bottles of mycoplasma broth medium containing arginine inoculated with cell samples are subcultured. Each bottle of mycoplasma broth medium is sub-cultivated separately to 2 bottles of mycoplasma semi-fluid medium containing arginine and 2 bottles of mycoplasma broth medium, and each bottle of mycoplasma medium containing arginine is sub-cultivated to 2 bottles of mycoplasma semi-fluid medium containing arginine and 2 bottles of mycoplasma broth medium containing arginine, each 1 mL inoculation volume, and cultured at 36° C.±1° C. for 21 days, and observed every 3 to 5 days.

### \textcircled{3} Endotoxin Detection

According to the Chinese Pharmacopoeia 2020 edition (volume IV) General Chapter <1143>, Bacterial Endotoxin Testing Method, the brief steps are as follows:

The endotoxin working standard (purchased from Zhanjiang A&C Biological Ltd.) is reconstituted with 1 mL of endotoxin testing water (purchased from Zhanjiang A&C Biological Ltd.), and gradient diluted after mixed by a vortex shaker for 15 min. The solution is mixed by a vortex shaker for 30 s in each dilution step, and finally it is diluted into 4λ and 2λ endotoxin standard solutions.

The cell suspension is diluted with endotoxin testing water, and mixed by a vortex shaker for 30 s in each dilution step. The dilution is used as the detected samples. The dilution ratio is not more than the Maximum Valid Dilution (MVD), which is calculated according to the formula MVD=C*L/λ, where L is the endotoxin limit of the sample, C is the concentration of the detected samples, and k is the sensitivity of the Limulus reagent.

One detected sample is added to 4λ endotoxin standard solution at a volume ratio of 1:1 as the endotoxin positive control.

8 bottles of Limulus reagents (purchased from Zhanjiang A&C Biological Ltd.) are reconstituted with 0.1 mL of endotoxin testing water respectively. 0.1 mL of the endotoxin positive control is added to 2 bottles of Limulus reagents as a parallel set of positive control of cells (PPC). 0.1 mL of 2λ endotoxin standard solution is added to 2 bottles of Limulus reagents as a parallel set of positive control (PC). 0.1 mL of endotoxin testing water is added to 2 bottles of Limulus reagents as a parallel set of negative control (NC). 0.1 mL of cell solution is added to 2 bottles of Limulus reagents with a dilution ratio not exceeding MVD, as a parallel set of cell detection.

A reaction tube is put into the preheated bacterial endotoxin tester and the countdown of 60 minutes is started. The reaction tube is taken out 1 minute before the end of 60 minutes and the results are observed and recorded.

The results are shown in Table 1. After 14 days, there is no bacterial grown in the cultivator containing the mesenchymal stromal cell samples, so the sterility test results of D1M1-P3, D1M2-P3, D1M1-P5 and D1M2-P5 are eligible. The mycoplasma tests of D1M1-P3, D1M2-P3, D1M1-P5 and D1M2-P5 are negative, so the test results are eligible. The endotoxin tests of D1M1-P3, D1M2-P3, D1M1-P5 and D1M2-P5 are negative, so the test results are eligible.

TABLE 1

| Name of multipotent | | | |
|---|---|---|---|
| mesenchymal stromal | Sterility | Mycoplasma | Endotoxin |
| cell samples | test | test | test |
| D1M1-P3 | negative | negative | negative |
| D1M2-P3 | negative | negative | negative |
| D1M1-P5 | negative | negative | negative |
| D1M2-P5 | negative | negative | negative |

### 2. Phenotypic Analysis

The expression of multipotent mesenchymal stromal cells-specific surface markers CD73, CD90, CD105, CD11b, CD19, CD34, CD45 and HLA-DR (referred to M. Dominici et al., Minimal criteria for defining multipotent mesenchymal stromal cells, The International Society for Cellular Therapy position statement, Cytotherapy (2006) Vol. 8, No. 4, 315-317) on hADSCs samples are analyzed by flow cytometry, and the steps are as follows:

The hADSCs samples of passages 3 or 5 are digested with Tryple™-Express (1×) at 37° C. for 2-3 min, and a volume of PBS (1×) which is more than 3 times the volume of Tryple™-Express (1×) is added to stop the digestion when cells become round and fall off. The cell suspension is pipetted into a 50 mL centrifuge tube and centrifuged at 300 g for 5 min. Washed twice with PBS (1×), cells are resuspended to a viable cell density of (0.5−1)×10⁷ cells/mL for future use.

100 μL of cell suspension is pipetted into a flow tube and incubated with 5 μL of pre-labelled antibodies (FITC-labeled anti-human CD34 antibody, FITC-labeled anti-human CD45 antibody, FITC-labeled anti-human CD11b antibody, FITC-labeled anti-human HLA-DR antibody, FITC-labeled anti-human CD73 antibody, FITC-labeled anti-human CD90 antibody, APC-labeled anti-human CD19 antibody, and PE-labeled anti-CD105 antibody) in the dark for 15 min at room temperature. FITC-labeled mouse IgG1, APC-labeled mouse IgG1, and PE-labeled mouse IgG1 are added as control groups. The used antibodies are purchased from Biolegend.

2 mL of sheath fluid is added to each flow tube, vortexed and centrifuged at 300 g for 5 min to discard the supernatant. Cells are resuspended with 300 μL of PBS (1×) containing 1% paraformaldehyde and analyzed using a flow cytometry.

The results are shown in Table 2. The expression of positive markers CD73, CD90, and CD105 on the surface of D1M1-P3, D1M2-P3, D1M1-P5 and D1M2-P5 are higher than 95%, and the expression of negative markers CD11b, CD19, CD34, CD45 and HLA-DR on the surface of D1M1-P3, D1M2-P3, D1M1-P5 and D1M2-P5 are less than 2%.

TABLE 2

| Name of multipotent | Expression rate of positive | | | Expression rate of negative | | | | |
|---|---|---|---|---|---|---|---|---|
| mesenchymal | markers | | | markers | | | | |
| stromal cell sample | CD73 | CD90 | CD105 | CD11b | CD19 | CD34 | CD45 | HLA-DR |
| D1M1-P3 | 98.02% | 99.86% | 99.68% | 0.37% | 0.12% | 0.45% | 0.72% | 0.28% |
| D1M2-P3 | 99.05% | 99.76% | 99.58% | 0.23% | 0.06% | 0.07% | 0.03% | 0.10% |
| D1M1-P5 | 98.08% | 99.93% | 99.94% | 0.34% | 0.00% | 1.21% | 0.60% | 0.07% |
| D1M2-P5 | 97.79% | 99.28% | 99.50% | 0.10% | 0.08% | 0.04% | 0.09% | 0.10% |

### 3. Cell Activity Detection

### \textcircled{1} Cell Viability Analysis

The cell suspension is diluted with 0.9% saline and mixed thoroughly with 0.4% trypan blue staining solution at a volume ratio of 9:1. 10 μL of mixture is pipetted into the counting chamber of a counting plate. Under a 10× objective lens, the total number of live cells and dead cells in the four squares are recorded respectively. The cell viability is calculated according to the following formula:

Cell viability (%)=total number of live cells/(total number of live cells+total number of dead cells)×100%

The cell viability of D1M1-P3, D1M2-P3, D1M1-P5, and D1M2-P5 all are more than 80%.

### \textcircled{2} Determination of Cell Growth Kinetics

When the confluence of hADSCs samples of passages 5 reaches 80%-90%, Tryple™-Express (1×) is used to digest the cells. Cells are adjusted with medium to the density of 3.2×10⁵/mL, 1.6×10⁵/mL, 0.8×10⁵/mL, 0.4×10⁵/mL, 0.2×10⁵/mL and 0.1×10⁵/mL, and seeded on a 96-well microplate at 100 μL/well. 100 μL of complete medium is added to the control well. Each group is set up with 6 duplicate wells.

After incubating at 37° C., 5% CO₂ for 4 hours, the culture medium in each well is discarded, and the CCK8 solution (DMEM/F12 (without phenol red):CCK8 (v/v)=100:10) is added into each well at 110 μL/well, and incubated at 37° C., 5% C02 for 2 hours.

The optical density at the wavelength of 450 nm (OD450) is measured using a multifunction microplate reader. Normalized by the average OD450 in the control well, the ΔOD450 values of the wells with different cell densities are obtained. A linear regression curve with ΔOD450 as the horizontal axis and the cell number as the longitudinal axis is fitted.

In a parallel experiment, hADSCs samples of passages 5 are plated in 96-well microplates at a density of 1×10⁴ cells/well. 100 μL of complete medium is added to the control well. Each group is set up with 6 duplicate wells, and 8 plates are prepared.

The cells are counted each day until the 8th day. The culture medium in each well is discarded, and the CCK8 solution (DMEM/F12 (without phenol red):CCK8 (v/v)=100:10) is added into each well at 110 μL/well, and incubated at 37° C., 5% C02 for 2 hours.

The OD450 is measured using a multifunction microplate reader. Normalized by the average OD450 in the control well, the ΔOD450 values of the wells are obtained. Based on the linear regression curve, the cell number of each well is calculated.

Growth curves are plotted using the mean values, and the population doubling time is calculated from the growth curve.

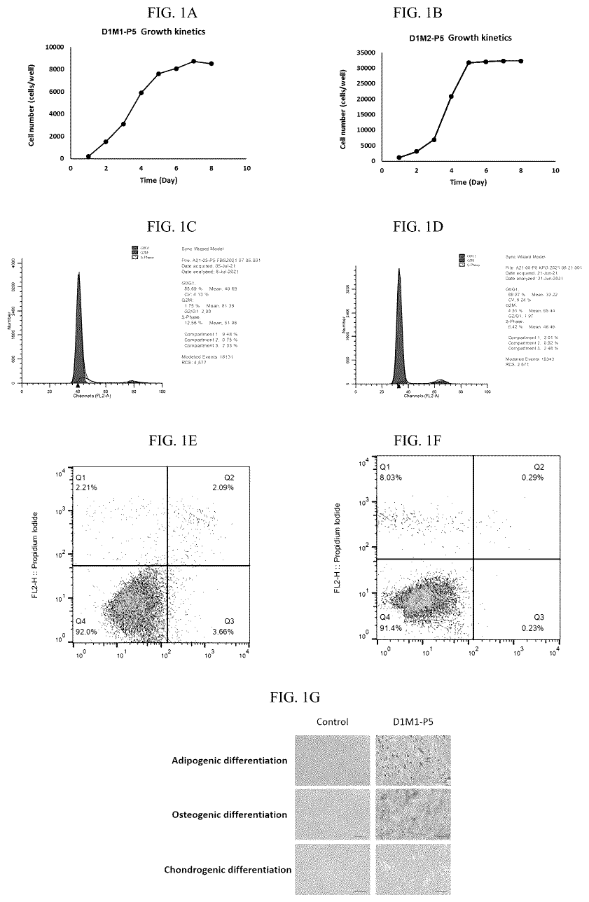

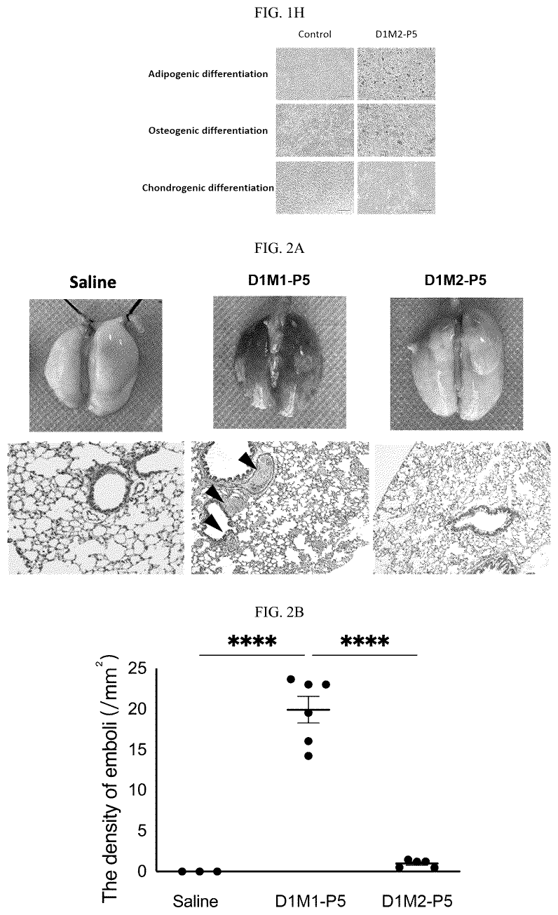

FIGS. 1A and 1B show the growth curves of D1M1-P5 and D1M2-P5, respectively. The hADSCs enter the logarithmic growth phase after 3 days of culture, enter the plateau phase after 6 days, and the cell amplification ability begins to decline after 7 days. The population doubling time of D1M1-P5 is 37.5 hours and that of D1M2-P5 is 21.9 hours.

### \textcircled{3} Cell Cycle Analysis

The hADSCs samples of passages 3 or 5 are digested with Tryple™-Express (1×) at 37° C. for 2-3 min, and centrifuged at 1000 rpm for 3-5 min. The supernatant is carefully discarded. The cell pellet is washed twice with 1 mL of pre-cooled PBS (1×) and resuspended to a density of 1×10⁶ cells/mL.

4 mL of pre-cooled 95% ethanol solution is vortexed with a low speed, with dropwise addition of 1 mL of cell suspension (operated on ice). Cells are mixed thoroughly and fixed at 4° C. for 2 hours or longer after mixing. Then, centrifugation is done at 1000 rpm for 3-5 min to precipitate the cells. Washed twice with 5 mL of pre-cooled PBS (1×), the cells are dispersed properly by gently tapping the bottom of the centrifuge tube to avoid cells aggregating.

Referring to Table 3, propidium iodide solution is prepared according to the number of samples to be tested, using the cell cycle and apoptosis detection kit (purchased from Beijing 4A Biotech Co., Ltd). Then 0.4 mL of propidium iodide solution is added to the cell samples, and the cell precipitation is slowly resuspended and incubated at 37° C. for 30 min in the dark. After washing twice with PBS (1×), the cells are resuspended in PBS (1×), and the cell cycle is detected using a flow cytometry and completed within 24 hours.

TABLE 3

| Reagent | 1 sample | | 6 samples | | 12 samples | |
|---|---|---|---|---|---|---|
| Dyeing buffer | 0.4 | mL | 2.4 | mL | 4.8 | mL |
| Propidium iodide solution (25×) | 15 | μL | 90 | μL | 180 | μL |
| RNase A (2.5 mg/mL) | 4 | μL | 24 | μL | 48 | μL |

FIGS. 1C and 1D show the results of cell cycle analysis. It can be seen that the proportions of D1M1-P5 in G1, S and G2 phases are 85.69%, 12.56% and 1.75%, respectively, and the proportions of D1M2-P5 in G1, S and G2 phases are 89.07%, 6.42% and 4.51%, respectively.

### \textcircled{4} Apoptosis Detection

The hADSCs samples of passages 5 are digested with Tryple™-Express (1×) at 37° C. for 2-3 min, and centrifuged at 1000 rpm for 3-5 min. The supernatant is carefully discarded, and the cell pellet is resuspended with 0.8 mL of 1×Binding Buffer (purchased from Beijing 4A Biotech Co., Ltd).

200 μL of hADSCs samples with a density of (2−5)×10⁵/mL is added to each flow tube, and incubated with 5 μL of Annexin-V-FITC in the dark for 10 min. After centrifugation, the cells are resuspended in 200 μL of binding buffer, then incubated with 5 μL of Propidium Iodide before flow cytometer analysis.

Results are shown in FIGS. 1E and 1F. The cell viability and the apoptosis rate of D1M1-P5 are 92.0% and 5.75%, respectively, and the cell viability and the cell apoptosis rate of D1M2-P5 are 91.4% and 0.52%, respectively.

### 4. Biological Activity Analysis

### \textcircled{1} Adipogenic Differentiation

Before the experiment, Solution A and Solution B are prepared according to the instructions of OriCell kit for human adipose-derived mesenchymal stem cell adipogenic differentiation (purchased from Cyagen Biosciences Inc., Cat #HUXMD-90031), and the following steps are performed:

Cells are seeded in a 6-well plate at a density of 2×10⁴ cells/cm², and 2 mL of complete medium is added to each well. The cells are cultured at 37° C. in 5% CO₂ until the cell confluence reaches 100%.

The culture supernatant is discarded. Cells are incubated in 2 mL of Solution A for 3 days, and switched to 2 mL of Solution B for 24 hours. After repeating 3 times, the cells are cultured continually in Solution B for 4-7 days until the lipid droplets become large and round enough.

The cells are washed and fixed using 4% paraformaldehyde solution and stained with 0.5% Oil Red 0 at room temperature for 20 min. After PBS (1×) washing for three times, images are taken using an inverted phase-contrast microscope.

### \textcircled{2} Osteogenic Differentiation

Before the experiment, the osteogenic medium is prepared according to the instructions of OriCell kit for human adipose-derived mesenchymal stem cell osteogenic differentiation (purchased from Cyagen Biosciences Inc., Cat #HUXMD-90021), and the following steps are performed:

Cells are seeded in a 6-well plate at a density of 2×10⁴ cells/cm², and 2 mL of complete medium is added to each well. The cells are cultured at 37° C. in 5% CO₂ until the cell confluence reaches 80-90%.

The culture supernatant is discarded. Cells are incubated in 2 mL of osteogenic medium, and the osteogenic medium is replaced every 3 days for 2-4 weeks, at which time a significant calcium deposit is observed under inverted microscope.

The cells are washed and fixed in 4% paraformaldehyde solution and stained by Alizarin Red S at room temperature for 5 min. After PBS (1×) washing for three times, images are taken using an inverted phase-contrast microscope.

### \textcircled{3} Chondrogenic Differentiation

Before the experiment, the chondrogenic medium is prepared according to the instructions of OriCell kit for human adipose-derived mesenchymal stem cell chondrogenic differentiation (purchased from Cyagen Biosciences Inc., Cat #HUXMD-90041), and the following steps are performed:

0.1% gelatin is added to a 6-well plate, shaken gently to cover the bottom of the well, and stand for 30 minutes. The gelatin is discarded, and the plate is dried.

Cells of passages 5 are inoculated into the 0.1% gelatin-coated 6-well plate at a density of 1×10⁴ cells/cm², and 2 mL of complete medium is added to each well. The cells are cultured at 37° C. and 5% CO₂ until the cell confluence reaches 80-90%.

The culture supernatant is discarded. Cells are induced in 2 mL of fresh chondrogenic medium (with 20 μL of TGF-β3), and the chondrogenic medium is replaced every 2-3 days for 2 weeks. The control wells are continuously cultured with complete medium.

The cells are washed and fixed in 4% paraformaldehyde solution and stained with Alcian Blue at room temperature for 30 min. After PBS (1×) washing for three times, images were taken using an inverted phase-contrast microscope.

FIGS. 1G and 1H illustrate the differentiation of multipotent mesenchymal stromal cells in in-vitro environment. The multipotent mesenchymal stromal cells are induced adipogenic differentiation, osteogenic differentiation and chondrogenic differentiation. The results shows that D1M1-P5 and D1M2-P5 are successfully induced into adipocytes, osteoblasts and chondroblasts, respectively.

Based on the above results, it shows that the P3 and P5-generation mesenchymal stromal cells cultured in different media are multipotent mesenchymal stromal cells, which meet the consented criteria for quality control of mesenchymal stromal cells.

### Embodiment 3. Animal Treatment by Multipotent Mesenchymal Stromal Cells

### 1. In-Vivo Infusion of Multipotent Mesenchymal Stromal Cells

6-8 week old male NCG mice (purchased from Gempharmatech Co., Ltd) are randomly assigned into groups. After the mice are fixed, the injection sites are sterilized, and the hADSCs samples of passages 5 (D1M1-P5 or D1M2-P5) which passed the quality control are resuspended in 0.9% saline and infused into each mouse via tail veins slowly, with an infusion dose of 1×10⁶ cells/mouse. The control group is infused with 0.9% saline.

After the infusion, the survival rate of the mice within 3 min is recorded. It is observed that the 6 mice that are infused with D1M1-P5 all died within 3 minutes, and the 6 mice that are infused with D1M2-P5 and the 6 mice that are infused with 0.9% saline all survived within 3 minutes. After observation, mice are anesthetized with avertin and euthanized by cutting off the abdominal aorta.

### 2. Immunohistochemical Examination

### \textcircled{1} Collection

The skin and muscle of the mouse are cut to expose the thoracic cavity. The right ventricle is punctured with a syringe, and 5 mL of 0.9% saline is slowly perfused throughout the body until the effluent liquid is no obvious blood color and relatively clear. The lung of mice is harvested immediately. The visual pathological observation is made, and the tissues are fixed in 10% formalin solution over 2 days.

### \textcircled{2} Hematoxylin-Eosin Staining

The obtained lung tissues undergo dehydration in gradient ethanol, embedded in paraffin, sectioned, stained with hematoxylin-eosin (HE) according to a general laboratory procedure. Finally, they are observed under a light microscope.

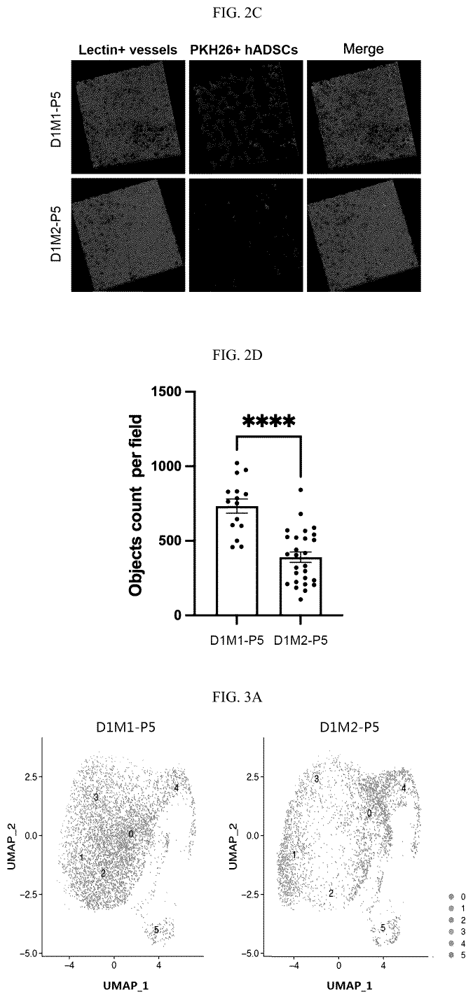

FIG. 2A shows the pathological results of lung after infusion with D1M1-P5, D1M2-P5 or saline into the mice. Compared with the control group, typical pulmonary congestion and severe pulmonary embolism symptom are observed in the mice infused with D1M1-P5. In contrast, D1M2-P5 does not cause any of the abovementioned adverse effects. It can also be seen from FIG. 2B that a significant number of venous clots develops in the lungs of the mice infused with D1M1-P5, and its emboli density is much higher than that of the D1M2-P5 group and the control group.

The D1M1-P5 or D1M2-P5 labeled with fluorescent PKH26 are further infused into mouse models, and the number of PKH26-positive cells in the lung is counted.

The results are shown in FIGS. 2C and 2D. A large amount of PKH26+D1M1-P5 is observed in the lungs of the thrombogenic mice, which is consistent with the immunohistochemical results, indicating that the mesenchymal stromal cells propagated under different culture conditions undergo different biological processes, or develop different lineages.

### Embodiment 4. Single-Cell RNA Sequencing

In order to identify the heterogeneity of mesenchymal stromal cells in different culture media, single-cell RNA sequencing is performed to detect the gene expression profile of mesenchymal stromal cells at the single-cell level. The steps are as follows:

### 1. Preparation of Single Cell Suspension

The hADSCs samples of passages 5 (D1M1-P5 and D1M2-P5) are diluted with Sample buffer to a cell suspension with a concentration of <1000 cells/μL. 1 μL of Calcein AM dye and 1 μL of Draq7 dye are added to 200 μL of cell suspension for cell staining.

The stained cell suspension is filtered with a 40 μm filter, and placed in the BD Rhapsody™ Scanner to detect the cell density and cell viability. According to the stock cell and buffer volumes obtained from the sample calculator function of the scanner, the cell suspension is diluted and prepared.

### 2. Single Cell Sorting

The diluted cell suspension is loaded on the Cartridge workflow that has two hundred thousand microwells (Cartridge Kit, purchased from BD Biosciences, Cat #633733), and cell loading and doublet rate are analyzed to evaluate the separation effect of single cells.

After unloaded cells are washed away, the BD Rhapsody beads are loaded on the Cartridge workflow, and bead & cell loading and doublet rate are analyzed to evaluate the number of beads bound to the single cell well.

After excess beads are washed away, the cell lysate is added to the Cartridge workflow for cell lysis. The mRNA content of each cell is captured by the probe via polyA/polyT on the surface of BD Rhapsody beads that have the same cell label (CL) and a variety of unique molecular identifier (UMI). The BD Rhapsody beads are recycled from the Cartridge workflow to a centrifuge tube.

3. Single-Cell cDNA Synthesis and Library Construction

Single-cell first-strand cDNA is reverse-synthesized and a library is constructed using Cartridge Reagent Kit (purchased from BD Biosciences, Cat #633731) and Whole Transcriptome Analysis (WTA) Amplification Kit (purchased from BD Biosciences, Cat #633801). The following operations are performed according to the instructions in the kits, which are briefly described as follows:

The recycled beads are washed, and reverse transcription reagents (Table 4) are added and mixed with the beads then incubated at 37° C. for 45 min.

TABLE 4

| Reagent | Addition per reaction system (μL) |
|---|---|
| Reverse transcription buffer | 40 |
| dNTPs (10 mM) | 20 |
| Dithiothreitol (DTT, 0.1M) | 10 |
| Additive (Bead RT/PCR Enhancer) | 12 |
| RNA enzyme inhibitor | 10 |
| Reverse transcriptase | 10 |
| Nuclease-free water | 98 |

Exonuclease is added, and incubated at 37° C. for 30 min and at 80° C. for 20 min, to remove probes that are not attached to mRNA on the surface of the beads.

Random primer mix (Table 5) is added, and incubated at 95° C. for 5 min, at 1200 rpm at 37° C. for 5 min, and at 1200 rpm at 25° C. for 15 min. Primer extension mix (Table 6) is added, incubated at 1200 rpm at 25° C. for 10 min, at 1200 rpm at 37° C. for 15 min, at 1200 rpm at 45° C. for 10 min, at 1200 rpm at 55° C. for 10 min, and the extended first-strand cDNA is eluted with the eluent without beads.

TABLE 5

| | Addition per |
|---|---|
| Reagent | reaction system (μL) |
| Extension buffer (WTA Extension Buffer) | 20 |
| Random primers (WTA Extension Primers) | 20 |
| Nuclease-free water | 134 |

TABLE 6

| Reagent | Addition per reaction system (μL) |
|---|---|
| dNTPs (10 mM) | 8 |
| Additive (Bead RT/PCR Enhancer) | 12 |
| WTA Extension Enzyme | 6 |

The product of random primer extension (RPE) is added in PCR amplified mixture containing universal primers and specific primers (Table 7) and amplified according to the procedure in Table 8. The amplified product is enriched and purified.

TABLE 7

| | Addition per |
|---|---|
| Reagent | reaction system (μL) |
| PCR buffer | 60 |
| Universal primer (Universal Oligo) | 10 |
| Specific primers (WTA Amplification Primer) | 10 |

TABLE 8

| Step | Cycle number | Temperature (° C.) | Time | |
|---|---|---|---|---|
| Initial Denaturation | 1 | 95 | 3 | min |
| Denaturation | 13 | 95 | 30 | s |
| Annealing | | 60 | 1 | min |
| Extension | | 72 | 1 | min |
| Final Extension | 1 | 72 | 2 | min |

| | | | |
|---|---|---|---|
| Hold | 1 | 4 | ∞ |

The amplified product is used as the template for PCR with whole transcriptome Index PCR amplified mixture (Table 9), and amplified according to the procedure in Table 10 (When the molar concentration of the amplified product is 1-2 nM, it is amplified by 9 cycles, and when the molar concentration of the amplified product is >2 nM, it is amplified by 8 cycles). The new amplified product is enriched and purified to obtain the single-cell sequencing library.

TABLE 9

| Reagent | Addition per reaction system (μL) |
|---|---|
| PCR buffer | 25 |
| Library Forward Primer | 5 |
| Library Reverse Primer | 5 |
| Nuclease-free water | 5 |

TABLE 10

| Step | Cycle number | Temperature (° C.) | Time | |
|---|---|---|---|---|
| Initial Denaturation | 1 | 95 | 3 | min |
| Denaturation | 8/9 | 95 | 30 | s |
| Annealing | | 60 | 30 | s |
| Extension | | 72 | 30 | s |
| Final Extension | 1 | 72 | 1 | min |

| | | | |
|---|---|---|---|
| Hold | 1 | 4 | ∞ |

### 4. Quality of Single-Cell Sequencing Library

The concentration of single-cell sequencing library is detected by Qubit instrument, and the fragment length of single-cell sequencing library is detected by Agilent 2100 bioanalyzer. It is found that the concentration of the library is 0.1-100 ng/μL, and the fragment length of the library is 460-550 bp.

### 5. Single-Cell Sequencing

The molar concentration of the single-cell sequencing library is calculated to be 1-100 nM based on the concentration and fragment length of the library. After diluted to the standard molar concentration 0.2-2 nM, the single-cell sequencing library is mixed with the sequencing control library PhiX of the same molar concentration based on the single-cell sequencing library: sequencing control library of 1: (0.05-0.5) for sequencing.

### 6. Quality of Sequencing Data

Sequencing data is analyzed by BD cw1-runner 3.1, and the quality of raw sequencing data is evaluated.

5095 D1M1-P5 and 3249 D1M2-P5 are sequenced with an average sequencing depth of 50 K/cell.

### Embodiment 5. Subpopulation Clustering

Raw sequencing data is converted to FASTQ format, and the quality of the sequencing data is analyzed. BD Rhapsody analysis pipeline v 1.9.1 (BD Biosciences) is used for cell barcode identification, read alignment, and UMI quantification with default parameters.

### 1. Data Preprocessing

Quality control: the sequences with read 1 length <60 and read 2 length <42, the sequences with base quality of read 1 and read 2<20, and the sequences with read 1 single nucleotide frequency (SNF)≥0.55 or read 2 SNF≥0.80 are filtered and removed.

Alignment and annotation: the valid reads after quality control are aligned to the human reference genome GRCh38, and the comparative results are annotated.

Gene expression matrix: expression read counts for each gene in all samples are collapsed and adjusted to unique molecular identifier (UMI) counts using recursive substitution error correction (RSEC). Putative cells are identified from background noise using second derivative analysis of all RSEC-adjusted UMI counts. The resulting output is a gene expression matrix with gene identities as columns and cell indices as rows.

### 2. Cell Filtration

RSEC-adjusted UMI count matrices are imported to R 4.1.0. and gene expression data analysis is conducted using the Seurat package 4.0.3. After identification of singlets, outlier cells are excluded from downstream analyses using the median absolute deviation (MAD) method. Cells with more than 3MAD from the median of mitochondria reads percentage, less than 3MAD from the median of expressed genes, or less than 3MAD from the median of UMI count are considered as outliers.

To eliminate confounding effects, such as cell cycle phases, sequencing depth and mitochondria percentage, Seurat is used to regress out the mentioned effects from analysis.

### 3. Dimensional Reduction

In order to obtain two-dimensional projections of the population's dynamics, Seurat's principal component analysis (PCA) is used to process the top 2000 highly variable genes in the normalized gene-barcode matrix, and the matrix is dimensionally reduced to obtain low-dimensional spatial information. Then, uniform manifold approximation and projection (UMAP) is performed to process the top 30 principal components (PCs) to realize cell visualization in two-dimensional space. The steps include:
Data is normalized by the NormalizeData function (normalization.method=“LogNormalize”);
The top 2000 genes ranked by variance as highly variable genes (HVGs) are selected by FindVariableFeature function (selection.method=“vst”, nfeatures=2000);
2000 highly variable genes are normalized by ScaleData function, and noise caused by cell cycle, etc. is removed;
Data is dimensionally reduced by the RunPCA function (features=VariableFeatures(object=adsc));
A neighbor graph is constructed by the shared nearest neighbor similarity algorithm (SNN) of the FindNeighbors function;
The parameters of the results of the SNN model are adjusted by the FindClusters function (resolution=0.1-1) to determine the number of cell subpopulation;
A visual dimensional reduction analysis is performed by the RunUMAP function.

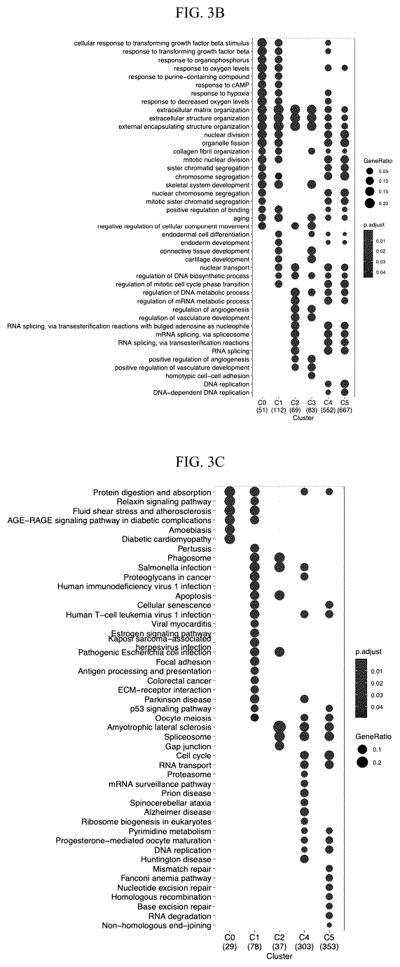

FIG. 3A shows the clustering results of cell subpopulation of D1M1-P5 and D1M2-P5, including 6 distinct clusters 0-5, and the proportions of each cluster in D1M1-P5 and D1M2-P5 are significantly different. As shown in FIGS. 3B and 3C, the expression of risk genes in each subpopulation is different, based on GO and KEGG to explore the risk genes of mesenchymal stromal cells. Thus, this indicates that mesenchymal stromal cells develop heterogeneity in different media, and the gene expression profiles of D1M1-P5 and D1M2-P5 are completely different.

### 4. Functional Clustering Analysis of Cell Subpopulation

To convert sparse gene expression matrix to pathway score matrix, all genes in the gene expression matrix are scored based on the canonical pathway. Then the pathway score matrix is subjected to dimensional reduction and visualization to obtain the functional clustering result of cell subpopulation. The schematic diagram is shown in FIG. 4. The steps are briefly described as follows:

By pathway enrichment analysis on the gene expression data of single cells, the scoring functions of ssGSEA, AUCell and Seurat are used to calculate the canonical pathway scores in each cells, and the pathway score matrix is obtained.

Then dimensional reduction and visualization are conducted based on the pathway score matrix as described above.

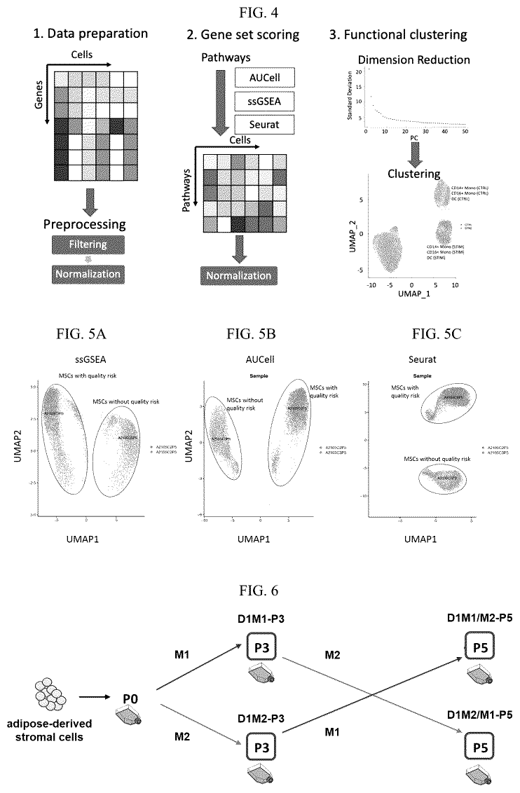

As shown in FIGS. 5A, 5B and 5C, the clustering results of three different scoring functions are the same. The cell subpopulations of D1M1-P5 (i.e. A2105C2P5) and D1M2-P5 (i.e. A2105C3P5) are distinguishably separated from each other. D1M1-P5 are all mesenchymal stromal cells with quality risk, and D1M2-P5 are all mesenchymal stromal cells without quality risk, indicating that significant functional changes exist after mesenchymal stromal cells are cultured in different media, which is consistent with the animal experiment results in Embodiment 2. The functional clustering procedure developed here should provide a valuable tool to identify specific functional subpopulations based on their transcriptomic profile.

According to the cell culture scheme in FIG. 6, the multipotent mesenchymal stromal cells from donor 1 are passaged from P0 to P3 generation using M1 or M2 medium, and then the medium is exchanged for subculture to P5 generation. The quality of the mesenchymal stromal cells of P3 and P5 generations is determined by the functional clustering analysis procedure.

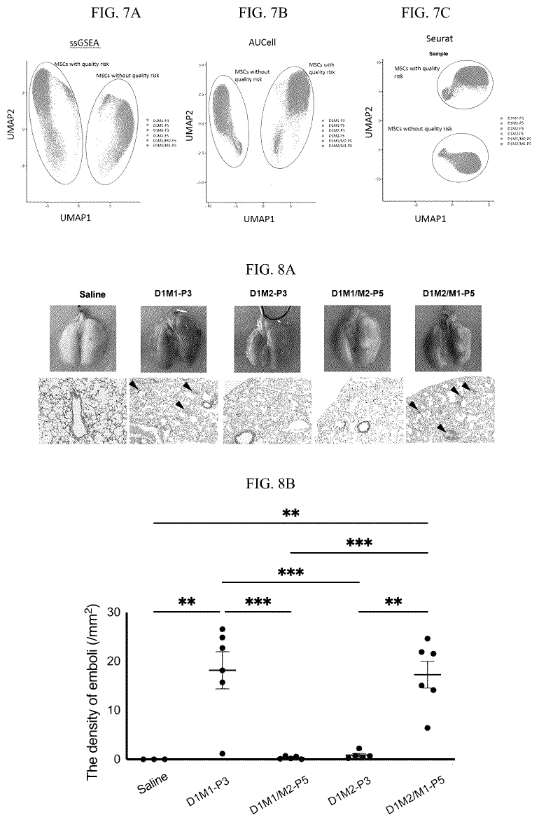

The results are shown in FIGS. 7A, 7B and 7C. The cell subpopulation of mesenchymal stromal cells cultured in M1 medium and mesenchymal stromal cells cultured in M2 medium are distinct clusters. The mesenchymal stromal cells cultured in M1 medium are all mesenchymal stromal cells with quality risk, and the mesenchymal stromal cells cultured in M2 medium are all mesenchymal stromal cells without quality risk. Mesenchymal stromal cells develop heterogeneity during their propagation under different culture conditions.

From the results of the animal experiments in FIGS. 8A and 8B, D1M1-P3 and D1M2/M1-P5 induce pulmonary embolism in mice, while D1M2-P3 and D1M1/M2-P5 do not induce pulmonary embolism in mice, which indicates the accuracy of the functional clustering results.

### Embodiment 6. Subpopulation Identification

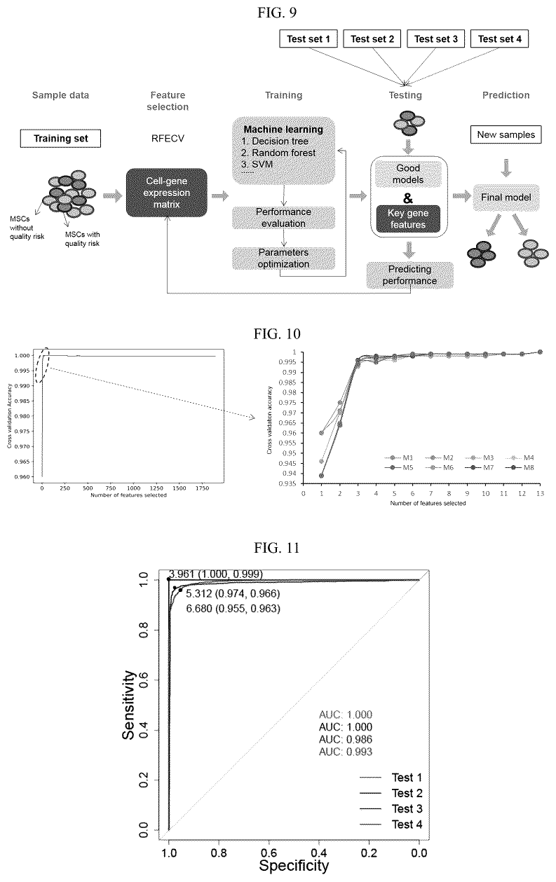

In this embodiment, a quality predictive model of mesenchymal stromal cells is constructed based on decision tree, random forest or support vector machine (SVM). The dataset is listed in Table 11. The schematic diagram is shown in FIG. 10. D1M1-P5 and D1M2-P5 represent the multipotent mesenchymal stromal cells obtained by subculture of primary multipotent mesenchymal stromal cells from donor 1, cultured in M1 or M2 medium to P5 generation, respectively. D1M1-P3 and D1M2-P3 represent the multipotent mesenchymal stromal cells obtained by subculture of primary multipotent mesenchymal stromal cells from donor 1, cultured in M1 or M2 medium to P3 generation, respectively. D2M1-P5 and D2M2-P5 represent the multipotent mesenchymal stromal cells obtained by subculture of primary multipotent mesenchymal stromal cells from donor 2, cultured in M1 or M2 medium to P5 generation, respectively. D2M3/M2-P5 represents the multipotent mesenchymal stromal cells obtained by subculture of primary multipotent mesenchymal stromal cells from donor 2, cultured in M3 medium (αMEM+5% Helios UltraGRO-Advanced) to P3 generation, and then cultured in M2 medium to P5 generation.

TABLE 11

| | Mesenchymal | | Functional clustering result of |
|---|---|---|---|
| Dataset | stromal cell | Gene expression data | cell subpopulation |
| Training | D1M1-P5 | randomly selective 70% single-cell | Mesenchymal stromal cell with |
| set | | gene expression data | quality risk |
| | D1M2-P5 | randomly selective 70% single-cell | Mesenchymal stromal cell |
| | | gene expression data | without quality risk |
| Test set | D1M1-P5 | remaining 30% single-cell gene | Mesenchymal stromal cell with |
| 1 | | expression data | quality risk |
| | D1M2-P5 | remaining 30% single-cell gene | Mesenchymal stromal cell |
| | | expression data | without quality risk |
| Test set | D1M1-P3 | single-cell gene expression data | Mesenchymal stromal cell with |
| 2 | | | quality risk |
| | D1M2-P3 | single-cell gene expression data | Mesenchymal stromal cell |
| | | | without quality risk |
| Test set | D2M1-P5 | single-cell gene expression data | Mesenchymal stromal cell with |
| 3 | | | quality risk |
| | D2M2-P5 | single-cell gene expression data | Mesenchymal stromal cell |
| | | | without quality risk |
| Test set | D2M1-P5 | single-cell gene expression data | Mesenchymal stromal cell with |
| 4 | | | quality risk |
| | D2M3/M2-P5 | single-cell gene expression data | Mesenchymal stromal cell |
| | | | without quality risk |

The steps are as follows:

### 1. Initial Hyperparameter

In the random forest model, the estimator (n_estimator) is set as 100, 200, 300, 400, 500, 600, 700, 800, 900, or 1000, and the maximum tree depth (max_depth) is 3, 5, or 7. In the SVM model, the regularization parameter C is 0.2, 0.6, 0.8, 1.0, 1.2, 1.6, 2.0, 2.2, 2.6 and 3.0, and the kernel parameter (kemel) is linear, “poly”, “rbf” or “sigmoid”.

### 2. Feature Selection

The importance of each gene of the training set in distinguishing mesenchymal stromal cells with quality risk from mesenchymal stromal cells without quality risk is ranked by using a machine learning method of recursive feature elimination with cross-validation (RFECV).

Starting from the most important gene, one gene is added successively and 10-fold cross-validation accuracy is calculated to determine the appropriate feature genes and the number of feature genes.

As shown in FIG. 11, selecting one most important gene as the feature gene, the 10-fold cross-validation accuracy of the models with different regularization parameter C on the training set reaches more than 94%. Selecting 13 most important genes (TAGLN, EFEMP1, TPM1, CLU, PTX3, IER3, IGFBP7, MFAP5, IL6, LUM, SERPINE2, CRIM1, and RHOB) as the feature genes, the 10-fold cross-validation accuracy of models with different regularization parameter C on the training set reaches 100%.

### 3. Model Derivation

Linear SVM models are developed using the 13 feature genes, and the model coefficient matrix (model weight matrix) is optimized by cross-validation to represent the importance score of the feature gene.

The model is trained by using test set 1, test set 2, test set 3, and test set 4 respectively, and the regularization parameter C is adjusted according to the prediction accuracy.

According to the test results shown in Table 12, the linear SVM model with C=0.0005 has the best performance on all test sets, which is selected as the quality predictive model of mesenchymal stromal cells.

TABLE 12
The prediction accuracy of the models with different regularization
parameters on the training set and the four test sets

| Regularization | Training | Test | Test | Test | Test |
|---|---|---|---|---|---|
| parameter C | set | set 1 | set 2 | set 3 | set 4 |
| 3 | 1.00 | 1.00 | 1.00 | 0.97 | 0.72 |
| 2.4 | 1.00 | 1.00 | 1.00 | 0.97 | 0.72 |
| 1.8 | 1.00 | 1.00 | 1.00 | 0.97 | 0.72 |
| 1.2 | 1.00 | 1.00 | 1.00 | 0.97 | 0.72 |
| 0.6 | 1.00 | 1.00 | 1.00 | 0.97 | 0.72 |
| 0.2 | 1.00 | 1.00 | 1.00 | 0.97 | 0.72 |
| 0.05 | 1.00 | 1.00 | 1.00 | 0.97 | 0.76 |
| 0.02 | 1.00 | 1.00 | 1.00 | 0.97 | 0.77 |
| 0.008 | 1.00 | 1.00 | 1.00 | 0.95 | 0.82 |
| 0.004 | 1.00 | 1.00 | 1.00 | 0.96 | 0.83 |
| 0.002 | 1.00 | 1.00 | 1.00 | 0.96 | 0.83 |
| 0.001 | 1.00 | 1.00 | 1.00 | 0.96 | 0.84 |
| 0.0005 | 1.00 | 1.00 | 1.00 | 0.97 | 0.84 |
| 0.0001 | 1.00 | 1.00 | 1.00 | 0.95 | 0.84 |

The prediction results of different types of mesenchymal stromal cells by the determined quality predictive model of mesenchymal stromal cells (SVM model, model complexity parameter C=0.0005) are shown in Table 13, with good test accuracy, precision, recall and score on four test sets. The determined 13 feature genes and their corresponding weight coefficient are shown in Table 14.

TABLE 13

| | Test set 1 | | Test set 2 | | Test set 3 | | Test set 4 | |
|---|---|---|---|---|---|---|---|---|
| | Mesenchymal | Mesenchymal | Mesenchymal | Mesenchymal | Mesenchymal | Mesenchymal | Mesenchymal | Mesenchymal |
| | stromal cell | stromal cell | stromal cell | stromal cell | stromal cell | stromal cell | stromal cell | stromal cell |
| Evaluation | with quality | without | with quality | without | with quality | without | with quality | without |
| indicator | risk | quality risk | risk | quality risk | risk | quality risk | risk | quality risk |
| Accuracy | 1.00 | | 1.00 | | 0.97 | | 0.84 | |
| Precision | 1.00 | 1.00 | 1.00 | 1.00 | 0.97 | 0.97 | 0.70 | 1.00 |
| Recall | 1.00 | 1.00 | 1.00 | 1.00 | 0.98 | 0.95 | 1.00 | 0.74 |
| F1 score | 1.00 | 1.00 | 1.00 | 1.00 | 0.97 | 0.96 | 0.83 | 0.85 |
| Logarithmic | 0.002 | | 0.003 | | 0.132 | | 0.416 | |
| loss | | | | | | | | |

TABLE 14

| Feature gene | Weight coefficient |
|---|---|
| TAGLN | −0.133 |
| EFEMP1 | −0.116 |
| TPM1 | −0.115 |
| CLU | 0.130 |
| PTX3 | −0.098 |
| IER3 | −0.103 |
| IGFBP7 | −0.090 |
| MFAP5 | −0.092 |
| IL6 | −0.097 |
| LUM | 0.089 |
| SERPINE2 | −0.096 |
| CRIM1 | −0.081 |
| RHOB | −0.076 |

### 4. Quality Score of Mesenchymal Stromal Cells at the Single Cell Level

Quality score of mesenchymal stromal cells at the single cell level is calculated based on the expression of the identified 13 feature genes and the weight coefficient of each feature gene determined by the quality predictive model of mesenchymal stromal cells, to quantitatively define the quality risk of single mesenchymal stromal cell. The function is as follows:

$$quality score = 1 + {e}^{- {\sum }_{i = 1}^{n} {W}_{i} * {G}_{i}}$$

Gi is the expression of the ith feature gene in single mesenchymal stromal cell, Wi is the weight coefficient of the ith feature gene, and n is the number of the feature genes. A positive value of Wi indicates that the increase of the feature gene expression will promote the quality risk of mesenchymal stromal cells, and a negative value of Wi indicates that the increase of the feature gene expression will suppress the quality risk of mesenchymal stromal cells.

The performance of quality score of mesenchymal stromal cells in 4 test sets and quality score thresholds are evaluated by receptor operating characteristic (ROC) curves and area under the curve (AUC).

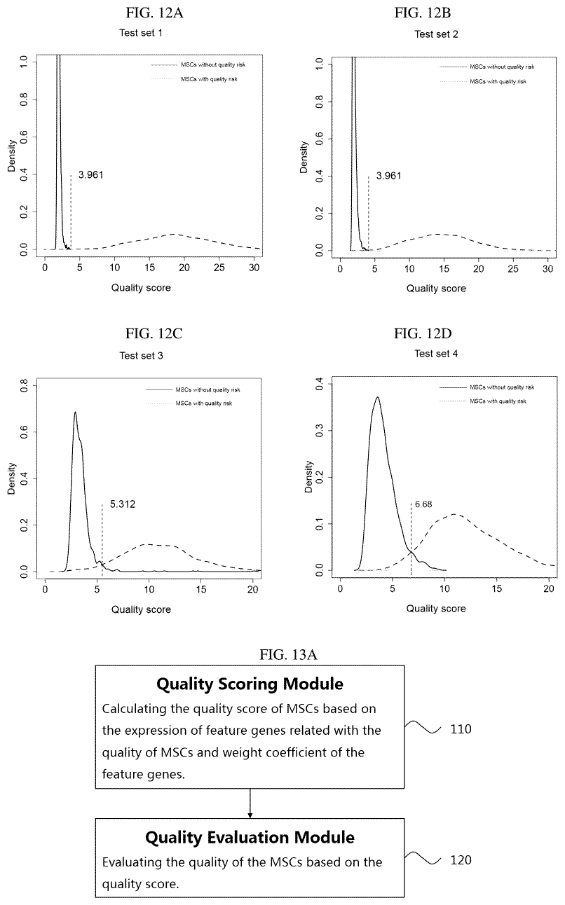

FIG. 12 shows the ROC curves and the corresponding AUCs of test set 1, test set 2, test set 3, and test set 4. The value of the highest point (with the highest sensitivity and specificity) of the ROC curve is used as the threshold for judging whether the mesenchymal stromal cells in the test set are the mesenchymal stromal cells with quality risk or the mesenchymal stromal cells without quality risk. From the ROC curve, the quality score thresholds of the 4 test sets are defined to be 3.961 (AUC=1), 3.961 (AUC=1), 5.312 (AUC=0.986) and 6.680 (AUC=0.993), respectively. The specific results are shown in FIGS. 12A, 12B, 12C and 12D.

### Embodiment 7. System for Evaluating the Quality of Mesenchymal Stromal Cells

In this embodiment, a system for evaluating the quality of mesenchymal stromal cells comprises an evaluation subsystem 10.

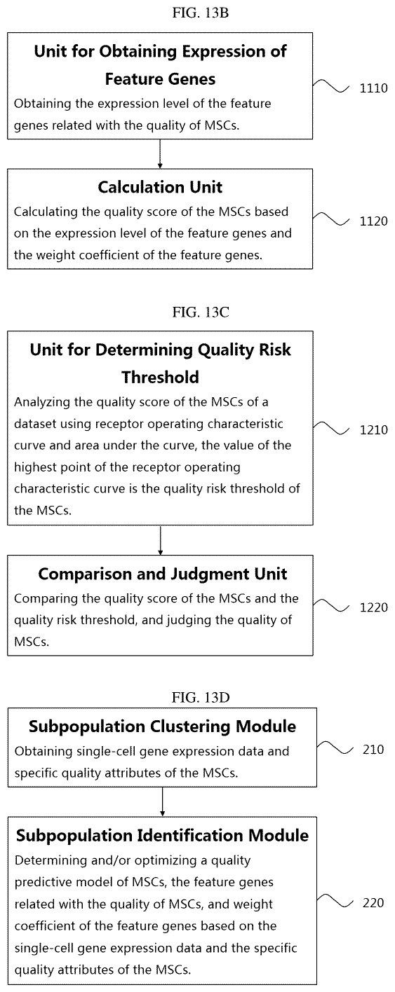

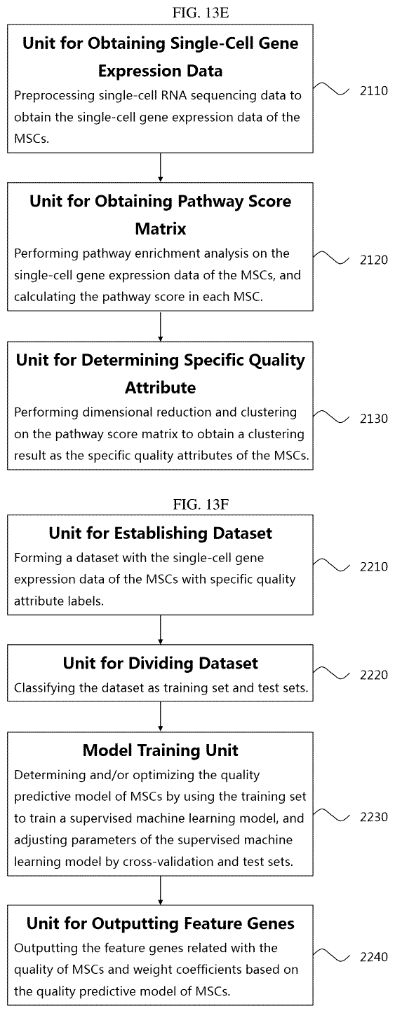

As shown in FIG. 13A, the evaluation subsystem 10 comprises:
a quality scoring module 110, used for calculating the quality score of mesenchymal stromal cells, based on the expression level of feature genes related with the quality of mesenchymal stromal cells and weight coefficient of the feature genes; and
a quality evaluation module 120, used for evaluating the quality of the mesenchymal stromal cells based on the quality score of the mesenchymal stromal cells.

As shown in FIG. 13B, the quality scoring module 110 comprises:
a unit 1110 for obtaining the expression level of the feature genes, used for obtaining the expression level of the feature genes related with the quality of mesenchymal stromal cells;
a calculation unit 1120, used for calculating the quality score of the mesenchymal stromal cells based on the expression level of the feature genes and the weight coefficient of the feature genes;
the function for calculating the quality score of the mesenchymal stromal cells is:

$$quality score = 1 + {e}^{- {\sum }_{i = 1}^{n} {W}_{i} * {G}_{i}}$$

Gi is the expression level of the ith feature gene, Wi is the weight coefficient of the ith feature gene, and n is the number of the feature genes.

As shown in FIG. 13C, the quality evaluation module 120 comprises:
a unit 1210 for determining a quality risk threshold of the mesenchymal stromal cells, used for analyzing the quality score of the mesenchymal stromal cells of a dataset using receptor operating characteristic curve and area under the curve, a value of the highest point of the receptor operating characteristic curve is the quality risk threshold of the mesenchymal stromal cells; wherein, the dataset contains the single-cell gene expression data of the mesenchymal stromal cells with known specific quality attribute labels;
a comparison and judgment unit 1220, used for comparing the quality score of the mesenchymal stromal cells and the quality risk threshold,
if the quality score of the mesenchymal stromal cells≥the quality risk threshold of the mesenchymal stromal cells, the mesenchymal stromal cells are the mesenchymal stromal cells with quality risk;
if the quality score of the mesenchymal stromal cells<the quality risk threshold of the mesenchymal stromal cells, the mesenchymal stromal cells are the mesenchymal stromal cells without quality risk.

The evaluation subsystem 10 comprises: a result output module, used for outputting a result report of the quality of the mesenchymal stromal cells.

The system for evaluating the quality of mesenchymal stromal cells further comprises an optimization subsystem 20.

As shown in FIG. 13D, the optimization subsystem 20 comprises:
a subpopulation clustering module 210, used for obtaining single-cell gene expression data and specific quality attributes of the mesenchymal stromal cells;
a subpopulation identification module 220, used for determining and/or optimizing a quality predictive model of mesenchymal stromal cells, the feature genes related with the quality of mesenchymal stromal cells, and weight coefficient of the feature genes based on the single-cell gene expression data and the specific quality attributes of the mesenchymal stromal cells.

As shown in FIG. 13E, the subpopulation clustering module 210 comprises:
a unit 2110 for obtaining single-cell gene expression data, used for preprocessing single-cell RNA sequencing data, to obtain the single-cell gene expression data of the mesenchymal stromal cells;
a unit 2120 for obtaining a pathway score matrix, used for performing pathway enrichment analysis on the single-cell gene expression data of the mesenchymal stromal cells, and calculating the pathway score in each mesenchymal stromal cell;
a unit 2130 for determining the specific quality attribute, used for performing dimensional reduction and clustering on the pathway score matrix to obtain a clustering result as the specific quality attributes of the mesenchymal stromal cells.

As shown in FIG. 13F, the subpopulation identification module 220 comprises:
a unit 2210 for establishing a dataset, used for forming a dataset with the single-cell gene expression data of the mesenchymal stromal cells with specific quality attribute labels;
a unit 2220 for dividing the dataset, used for classifying the dataset as training set and test sets;
a model training unit 2230, used for determining and/or optimizing the quality predictive model of mesenchymal stromal cells by using the training set to train a supervised machine learning model, and adjusting parameters of the supervised machine learning model by cross-validation and test sets;
a unit 2240 for outputting the feature genes, used for outputting the feature genes related with the quality of mesenchymal stromal cells and weight coefficients based on the quality predictive model of mesenchymal stromal cells.

### Embodiment 8. Using the System to Evaluate the Quality of Mesenchymal Stromal Cell Samples

According to the identified 13 feature genes and the weight coefficient determined by the quality predictive model of mesenchymal stromal cells (Table 14), the expression of the feature genes in D1M1/M2-P5 and D1M2/M1-P5 at the single cell level is detected by using the subsystem 10 of the system for evaluating the quality of mesenchymal stromal cells. Based on the function, quality scores of D1M1/M2-P5 and D1M2/M1-P5 are calculated to evaluate the quality of the mesenchymal stromal cell. The quality score threshold is 3.961.

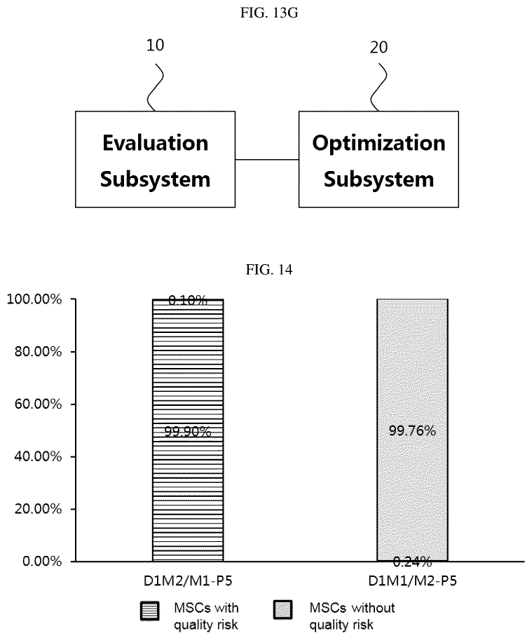

The result is shown in FIG. 14. It indicates that 99.90% of D1M2/M1-P5 are the mesenchymal stromal cells with quality risk and 0.10% of D1M2/M1-P5 are the mesenchymal stromal cells without quality risk, while 0.24% of D1M1/M2-P5 are the mesenchymal stromal cells with quality risk, and 99.76% of D1M1/M2-P5 are the mesenchymal stromal cells without quality risk. The predictive result is consistent with the functional clustering results of cell subpopulation in FIGS. 7A, 7B and 7C and the animal experiment outcomes in FIGS. 8A and 8B. It shows that the quality risk of mesenchymal stromal cells can be predicted accurately by the quality predictive model.

### Embodiment 9. Analysis Service Based on the System

When the user uploads single-cell RNA sequencing data of mesenchymal stromal cells to the analysis website, the quality scoring module 110 of the evaluation subsystem 10 in the system for evaluating the quality of mesenchymal stromal cells obtains the expression level of the feature genes related with the quality of mesenchymal stromal cells, and calculates the quality score of the mesenchymal stromal cells based on the expression level of the feature genes and the weight coefficient of the feature genes.

The function for calculating the quality score of the mesenchymal stromal cells is:

$$quality score = 1 + {e}^{- {\sum }_{i = 1}^{n} {W}_{i} * {G}_{i}}$$

Gi is the expression level of the ith feature gene, Wi is the weight coefficient of the ith feature gene, and n is the number of the feature genes.

The quality evaluation module 120 of the evaluation subsystem 10 compares the quality score of the mesenchymal stromal cells and the quality risk threshold;
if the quality score of the mesenchymal stromal cells≥the quality risk threshold of the mesenchymal stromal cells, the mesenchymal stromal cells are the mesenchymal stromal cells with quality risk;
if the quality score of the mesenchymal stromal cells<the quality risk threshold of the mesenchymal stromal cells, the mesenchymal stromal cells are the mesenchymal stromal cells without quality risk.

The result output module of the evaluation subsystem 10 outputs the quality evaluation report of the mesenchymal stromal cells.

When the administrator upgrades the analysis website, the subpopulation clustering module 210 of the optimization subsystem 20 in the system for evaluating the quality of mesenchymal stromal cells preprocesses the single-cell RNA sequencing data to obtain the single-cell gene expression data, performs pathway enrichment analysis on the single-cell gene expression data and calculates the pathway score in each mesenchymal stromal cell to obtain the pathway score matrix, and performs dimensional reduction and clustering on the pathway score matrix to obtain a clustering result as the specific quality attributes of the mesenchymal stromal cells.

The subpopulation identification module 220 of the optimization subsystem 20 integrates the single-cell gene expression data of mesenchymal stromal cells with specific quality attributes into the original dataset to form a new dataset, which is classified as training set and test sets, optimizes the quality predictive model of mesenchymal stromal cells using the training set and test sets, and outputs the feature genes related with the quality of mesenchymal stromal cells and weight coefficients.

The quality scoring module 110 of the evaluation subsystem 10 obtains the expression level of the feature genes related with the quality of the mesenchymal stromal cells, and calculates the quality score of the mesenchymal stromal cells based on the expression level of feature genes and weight coefficient of the feature genes.

The function for calculating the quality score of the mesenchymal stromal cells is:

$$quality score = 1 + {e}^{- {\sum }_{i = 1}^{n} {W}_{i} * {G}_{i}}$$

Gi is the expression level of the ith feature gene, Wi is the weight coefficient of the ith feature gene, and n is the number of the feature genes.

The quality evaluation module 120 of the evaluation subsystem 10 compares the quality score of the mesenchymal stromal cells and the quality risk threshold, wherein, the dataset contains the single-cell gene expression data of the mesenchymal stromal cells with known specific quality attributes;
if the quality score of the mesenchymal stromal cells≥the quality risk threshold of the mesenchymal stromal cells, the mesenchymal stromal cells are the mesenchymal stromal cells with quality risk;
if the quality score of the mesenchymal stromal cells<the quality risk threshold of the mesenchymal stromal cells, the mesenchymal stromal cells are the mesenchymal stromal cells without quality risk.

The result output module of the evaluation subsystem 10 outputs the quality evaluation report of the mesenchymal stromal cells.

The applicant declares that the present disclosure illustrates the detailed method of the present disclosure by the above-mentioned embodiments, but the present disclosure is not limited to the detailed method mentioned above, that is, it does not mean that the present disclosure must rely on the above-mentioned detailed method to be implemented. Those skilled in the art should understand that any improvement of the present disclosure, the equivalent replacement of each raw material of the product of the present disclosure, the addition of auxiliary components, the selection of specific methods, etc., all fall within the protection scope and the scope of the present disclosure.

1. A system for evaluating the quality of mesenchymal stromal cells, comprising:
a quality scoring module, used for calculating a quality score of the mesenchymal stromal cells, based on an expression level of feature genes related to the quality of the mesenchymal stromal cells and a weight coefficient of the feature genes; and
a quality evaluation module, used for evaluating the quality of the mesenchymal stromal cells based on the quality score of the mesenchymal stromal cells.

2. The system for evaluating the quality of mesenchymal stromal cells according to claim 1, wherein the quality scoring module comprises:
(1) a unit for obtaining the expression level of the feature genes, used for obtaining the expression level of the feature genes related with the quality of the mesenchymal stromal cells; and
(2) a calculation unit, used for calculating the quality score of the mesenchymal stromal cells based on the expression level of the feature genes and the weight coefficient of the feature genes; wherein
the function for calculating the quality score of the mesenchymal stromal cells is:
$$quality score = 1 + {e}^{- {\sum }_{i = 1}^{n} {W}_{i} * {G}_{i}}$$
in which Gi is an expression level of the ith feature gene, Wi is a weight coefficient of the ith feature gene, and n is a number of the feature genes.

3. The system for evaluating the quality of mesenchymal stromal cells according to claim 1, wherein the quality evaluation module comprises:
(1) a unit for determining a quality risk threshold of the mesenchymal stromal cells, used for analyzing the quality score of the mesenchymal stromal cells of a dataset using a receptor operating characteristic curve and an area under the curve, a value of a highest point of the receptor operating characteristic curve is a quality risk threshold of the mesenchymal stromal cells; wherein, the dataset contains single-cell gene expression data of the mesenchymal stromal cells with known specific quality attributes; and
(2) a comparison and judgment unit, used for comparing the quality score of the mesenchymal stromal cells and the quality risk threshold, wherein
if the quality score of the mesenchymal stromal cells≥the quality risk threshold of the mesenchymal stromal cells, the mesenchymal stromal cells are the mesenchymal stromal cells with quality risk; and
if the quality score of the mesenchymal stromal cells<the quality risk threshold of the mesenchymal stromal cells, the mesenchymal stromal cells are the mesenchymal stromal cells without quality risk.

4. The system for evaluating the quality of mesenchymal stromal cells according to claim 1, wherein the system for evaluating the quality of the mesenchymal stromal cells further comprises:
a result output module, used for outputting a result report of the quality of the mesenchymal stromal cells.

5. (canceled)

6. The system for evaluating the quality of mesenchymal stromal cells according to claim 1, wherein the mesenchymal stromal cells include any one or a combination of at least two of mesenchymal stromal cells, multipotent stromal cells, multipotent mesenchymal stromal cells and medicinal signaling cells.

7. The system for evaluating the quality of mesenchymal stromal cells according to claim 1, wherein the mesenchymal stromal cells include any one or a combination of at least two of adipose-derived mesenchymal stromal cells, umbilical cord mesenchymal stromal cells, placenta-derived mesenchymal stromal cells, bone marrow mesenchymal stromal cells, dental pulp mesenchymal stromal cells, menstrual blood-derived mesenchymal stromal cells, amniotic epithelial mesenchymal stromal cells, and bronchial basal cells.

8. An optimization subsystem, comprising:
a subpopulation clustering module, used for obtaining single-cell gene expression data and specific quality attributes of the mesenchymal stromal cells; and
a subpopulation identification module, used for determining and/or optimizing a quality predictive model of the mesenchymal stromal cells, the feature genes related with the quality of the mesenchymal stromal cells, and the weight coefficient of the feature genes based on the single-cell gene expression data and the specific quality attributes of the mesenchymal stromal cells.

9. The optimization subsystem according to claim 8, wherein the subpopulation clustering module comprises:
(1) a unit for obtaining the single-cell gene expression data, used for preprocessing single-cell RNA sequencing data, to obtain the single-cell gene expression data of the mesenchymal stromal cells;
(2) a unit for obtaining a pathway score matrix, used for performing pathway enrichment analysis on the single-cell gene expression data of the mesenchymal stromal cells, and calculating a pathway score for each mesenchymal stromal cell; and
(3) a unit for determining a specific quality attribute, used for performing dimensional reduction and clustering on the pathway score matrix to obtain a clustering result as the specific quality attributes of the mesenchymal stromal cells.

10. The optimization subsystem according to claim 8, wherein
the subpopulation identification module comprises:
(1) a unit for establishing a dataset, used for forming a dataset with the single-cell gene expression data of the mesenchymal stromal cells with specific quality attributes;
(2) a unit for dividing the dataset, used for classifying the dataset as a training set and test sets;
(3) a model training unit, used for determining and/or optimizing the quality predictive model of the mesenchymal stromal cells by using the training set to train a supervised machine learning model, and adjusting parameters of the supervised machine learning model by cross-validation and test sets; and
(4) a unit for outputting the feature genes, used for outputting the feature genes related to the quality of the mesenchymal stromal cells and the weight coefficient based on the quality predictive model of the mesenchymal stromal cells.

11. The optimization subsystem according to claim 8, wherein the supervised machine learning model comprises:
any of a perceptron model, a K-nearest neighbor algorithm, a naive Bayesian model, a decision tree model, a logical regression, a support vector machine, a random forest, a boosting method model, an EM algorithm and a conditional random field.

12. A method for establishing a quality predictive model of mesenchymal stromal cells, comprising:
obtaining single-cell gene expression data with specific quality attributes of the mesenchymal stromal cells to form a dataset, which is classified as a training set and test sets; and
determining a quality predictive model of the mesenchymal stromal cells by using the training set to train a supervised machine learning model and adjusting parameters of the supervised machine learning model by cross-validation and testing with the test sets.

13. The method for establishing a quality predictive model of mesenchymal stromal cells according to claim 12, wherein the obtaining single-cell gene expression data with specific quality attributes of the mesenchymal stromal cells comprises:
obtaining the single-cell gene expression data of the mesenchymal stromal cells by single-cell RNA sequencing of the mesenchymal stromal cells;
obtaining a pathway score matrix by pathway enrichment analysis on the single-cell gene expression data of the mesenchymal stromal cells, and calculating a pathway enrichment score for each of the mesenchymal stromal cells; and
obtaining a clustering result of the mesenchymal stromal cells as the specific quality attributes of the mesenchymal stromal cells by bioinformatic analysis on the pathway score matrix.

14. The method for establishing a quality predictive model of mesenchymal stromal cells according to claim 13, wherein the bioinformatic analysis on the pathway score matrix of the mesenchymal stromal cells comprises:
performing dimensional reduction and clustering on the pathway score matrix.

15. The system for evaluating the quality of mesenchymal stromal cells according to claim 2, wherein the quality evaluation module comprises:
(1) a unit for determining a quality risk threshold of the mesenchymal stromal cells, used for analyzing the quality score of the mesenchymal stromal cells of a dataset using a receptor operating characteristic curve and an area under the curve, a value of a highest point of the receptor operating characteristic curve is a quality risk threshold of the mesenchymal stromal cells; wherein, the dataset contains single-cell gene expression data of the mesenchymal stromal cells with known specific quality attributes; and
(2) a comparison and judgment unit, used for comparing the quality score of the mesenchymal stromal cells and the quality risk threshold, wherein
if the quality score of the mesenchymal stromal cells≥the quality risk threshold of the mesenchymal stromal cells, the mesenchymal stromal cells are the mesenchymal stromal cells with quality risk; and
if the quality score of the mesenchymal stromal cells<the quality risk threshold of the mesenchymal stromal cells, the mesenchymal stromal cells are the mesenchymal stromal cells without quality risk.

16. The system for evaluating the quality of mesenchymal stromal cells according to claim 2, wherein the system for evaluating the quality of the mesenchymal stromal cells further comprises:
a result output module, used for outputting a result report of the quality of the mesenchymal stromal cells.

17. The system for evaluating the quality of mesenchymal stromal cells according to claim 2, wherein the mesenchymal stromal cells include any one or a combination of at least two of mesenchymal stromal cells, multipotent stromal cells, multipotent mesenchymal stromal cells and medicinal signaling cells.

18. The system for evaluating the quality of mesenchymal stromal cells according to claim 1, wherein the mesenchymal stromal cells include any one or a combination of at least two of adipose-derived mesenchymal stromal cells, umbilical cord mesenchymal stromal cells, placenta-derived mesenchymal stromal cells, bone marrow mesenchymal stromal cells, dental pulp mesenchymal stromal cells, menstrual blood-derived mesenchymal stromal cells, amniotic epithelial mesenchymal stromal cells, and bronchial basal cells.

19. The optimization subsystem according to claim 9, wherein the subpopulation identification module comprises:
(1) a unit for establishing a dataset, used for forming a dataset with the single-cell gene expression data of the mesenchymal stromal cells with specific quality attributes;
(2) a unit for dividing the dataset, used for classifying the dataset as a training set and test sets;
(3) a model training unit, used for determining and/or optimizing the quality predictive model of the mesenchymal stromal cells by using the training set to train a supervised machine learning model, and adjusting parameters of the supervised machine learning model by cross-validation and test sets; and
(4) a unit for outputting the feature genes, used for outputting the feature genes related to the quality of the mesenchymal stromal cells and the weight coefficient based on the quality predictive model of the mesenchymal stromal cells.

20. The optimization subsystem according to claim 9, wherein the supervised machine learning model comprises:
any of a perceptron model, a K-nearest neighbor algorithm, a naive Bayesian model, a decision tree model, a logical regression, a support vector machine, a random forest, a boosting method model, an EM algorithm and a conditional random field.

21. The optimization subsystem according to claim 10, wherein the supervised machine learning model comprises:
any of a perceptron model, a K-nearest neighbor algorithm, a naive Bayesian model, a decision tree model, a logical regression, a support vector machine, a random forest, a boosting method model, an EM algorithm and a conditional random field.
